(19) United States
(12) Patent Application Publication
(10) Pub. No.: US 2023/0321229 A1
(43) Pub. Date: Oct. 12, 2023

(54) VACCINE COMPRISING AN ANTIGEN AND A TLR2 AGONIST

(71) Applicant: ISR IMMUNE SYSTEM REGULATION HOLDING AB (PUBL), Solna (SE)

(72) Inventors: Ola WINQVIST, Uppsala (SE); Johan SJÖDAHL, Solna (SE)

(21) Appl. No.: 18/024,213

(22) PCT Filed: Sep. 3, 2021
(86) PCT No.: PCT/EP2021/074399

§ 371 Date: Mar. 1, 2023

(30) Foreign Application Priority Data
Sep. 3, 2020 (EP) 20194425.3

(51) Int. Cl. A61K 39/39; A61K 31/07; A61K 31/59; A61K 31/7048; A61K 39/215; A61P 31/14

(52) U.S. Cl. CPC A61K 39/39; A61K 31/07; A61K 31/59; A61K 31/7048; A61K 39/215; A61P 31/14; A61K 2039/55511

(57) ABSTRACT

The present invention provides vaccine kits and a method for vaccination using such vaccination kits.

### FIELD OF THE INVENTION

The present invention relates to the fields of medical science, immunology and vaccines. The present invention provides vaccine kits and compositions capable of stimu-lating the immune system, e.g. against pathogenic bacteria and vira. The present invention also provides methods administration of the vaccines so that the individual ob-tains immunity from pathogenic bacteria and vira.

### BACKGROUND OF THE INVENTION

There is a need for effective and safe new vaccines for preventing diseases originating from bacterial and viral infections. There is also a need for new adjuvants and opti-mized administration to achieve a better immune response following vaccination. Such new vaccines are required to have attractive combinations of properties including strong immune response when formulated into a product and low toxicity. In particular there is a need for such new vaccines in the field of viral infections where several MERS and SARS outbreaks have spread through several countries just during the last two decades. Such epidemics may have severe impacts also beyond those individuals attracting the virus, e.g. on travelling, hospitals, businesses and society at large.

Hence, there is a need for effective and safe new vaccines for preventing diseases originating from bacterial and in particular viral infections.

### SUMMARY OF THE INVENTION

The present inventors have discovered that a vaccination method comprising an antigen and a TLR2 agonist as adjuvant has good effects against corona virus, in particular when combined with a vitamin, e.g. vitamin A.

In a first aspect the present invention provides a method for vaccination, wherein a composition comprising an antigen, a Toll-like receptor 2 (TLR2) agonist and at least one pharmaceutically acceptable excipient is for pulmonal or intranasal administration, and wherein vitamin A is orally administered at least once within three days before or after the administration of said composition.

In particular, it is believed that the pulmonal and intranasal administration promotes immunoglobulin switch towards IgA, the immunoglobulin specialized for mucosal surfaces including the lung and gut. The TLR2 agonist is believed to promote activation of mac-rophages resulting in increased antigen presenting capacity, increased expression of costimulatory molecules including CD86 in addition to the increased production and re-lease of cytokines and chemokines including interferons. Thus, the TLR2 agonist promotes T cell activation, the foundation for the successful induction of a productive neu-tralizing B cell response.

In a second aspect the present invention provides a vaccine kit comprising
a composition comprising an antigen, a TLR2 agonist and at least one pharmaceutically acceptable excipient, and
a label informing that said composition is to be used for vaccination by co-administration of vitamin A.

In a third aspect the present invention provides a vaccine kit comprising
a first composition comprising an antigen, a TLR2 agonist and at least one pharmaceutically acceptable excipient, and
a second composition comprising vitamin A.

In one embodiment the TLR2 agonist is the compound of Formula (I):

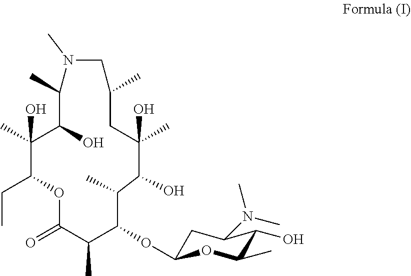

or a pharmaceutically acceptable salt thereof.

In another embodiment the antigen is a protein or a multimer thereof, a peptide or a multimer thereof, an attenuated bacterium or an attenuated virus. Multimers of a protein or peptide mean that at least two proteins or peptides are covalently linked to form dimers, trimers, tetramers etc. Such multimers may have better antigen properties.

In another embodiment the antigen is attenuated SARS-Cov-2 or a component thereof.

In another embodiment the antigen is the spike protein from SARS-Cov-2 or a part thereof.

In addition, in comparison with known vaccine compositions, the method for vaccination according to the present invention shows improved properties for effectively raising an immune response following vaccination of an individual, thus provide a better protection against future bacterial or viral challenges.

### BRIEF DESCRIPTION OF THE DRAWINGS

FIG. 1. A pleiotropic role of vitamin A in regulating adaptive immunity for SIgA production. This is a conceptual view for the production of IgA by the adaptive immune system, showing the main steps known to be regulated by vitamin A. The vitamin is in-volved in practically all steps along the production line, from the antigen uptake to sIgA secretion in the lumen. The main mechanisms shown here include educating mucosal DCs (CD103+DC) to synthesize retinoic acid via upregulating the expression of RALDH enzyme for converting VA to RA, imprinting T and B cells with gut-homing re-ceptors (a4β7 integrin and chemokine receptor CCR9), differentiation of T cells into various regulatory and effector T cell subsets, polarizing B cells in favor of IgA+antibody secreting cells (IgA+ASCs) and finally transport the complete sIgA molecule across the epithelial cells for secretion at the apical surface. Heavy black arrows pointing upward indicate the subsets promoted by RA (Treg, Th2, B cell, and (IgA+ASCs), whereas the dash thin arrow pointing downward represents its blocking action on Th17 development.

FIG. 2. IgG titres against Spike (Wuhan), RBD (South Africa) and RBD (UK) following immunisation. Samples were acquired on Day 28. Immunisation was performed on Day 0 and 14. Data is presented as geometric mean±geometric SD. ID 366 (Group 6) was excluded.

FIG. 3. IgA titres against Spike (Wuhan), RBD (South Africa) and RBD (UK) following immunisation. Samples were acquired on Day 28. Immunisation was performed on Day 0 and 14. Data is presented as geometric mean±geometric SD. ID 366 (Group 6) was excluded.

FIG. 4. IgG titres against Spike (Wuhan), RBD (South Africa) and RBD (UK) in BAL. Samples were acquired at termination. Immunisation was performed on Day 0 and 14. Data is presented as geometric mean±geometric SD. ID 366 (Group 6) was excluded.

FIG. 5. IgA titres against Spike (Wuhan), RBD (South Africa) and RBD (UK) in BAL. Samples were acquired at termination. Immunisation was performed on Day 0 and 14. Data is presented as geometric mean±geometric SD. ID 366 (Group 6) was excluded.

FIG. 6. Anti-IgG in sera of the individual immunised mice from the study of Example 3.

FIG. 7. Averaged Anti-IgG from the immunized groups of mice from the study of Example 3.

### DESCRIPTION OF THE INVENTION

Immunoglobulin A (IgA), one of the five primary immunoglobulins, plays a pivotal role in mucosal homeostasis in the gastrointestinal, respiratory, and genitourinary tracts, func-tioning as the dominant antibody of immunity in this role. It is the second most abun-dant immunoglobulin type found in the body and, consequently, has a crucial role in protection against antigens.

IgA gets produced by class switching of Ig, which is regulated by various processes. The binding of CD40-CD40L and secretion of other cytokines IL-4, IL-5, IL-6, IL-10, and IL-21 promote maturation of Th2 cells, which promote class switching to different Ig subtypes. Retinoic acid, a metabolite of vitamin A, synergistically acts with IL-5 and IL-6 to induce IgA secretion as well.

Vitamin A (retinoid) is a micronutrients known to be required in trace amounts in the diet of practically all vertebrate animals, as it cannot be synthesized in sufficient quanti-ties to maintain physiological health. High concentrations can have some therapeutic effects, as the vitamin A and its metabolites are known to have adjuvant activity.

The retinol must be oxidized to retinal by intracellular enzyme alcohol dehydrogenase (ADH) prior to being irreversibly catabolized by retinal dehydrogenase (RALDH) to its biologically active form all-trans-retinoic acid (from now referred to as RA). This bioac-tive metabolite can be synthesized by many cell types and tissues known to possess the RALDH enzyme necessary for such a conversion, including DCs from different tissues, e.g., gut, lungs, skin and their draining lymph nodes.

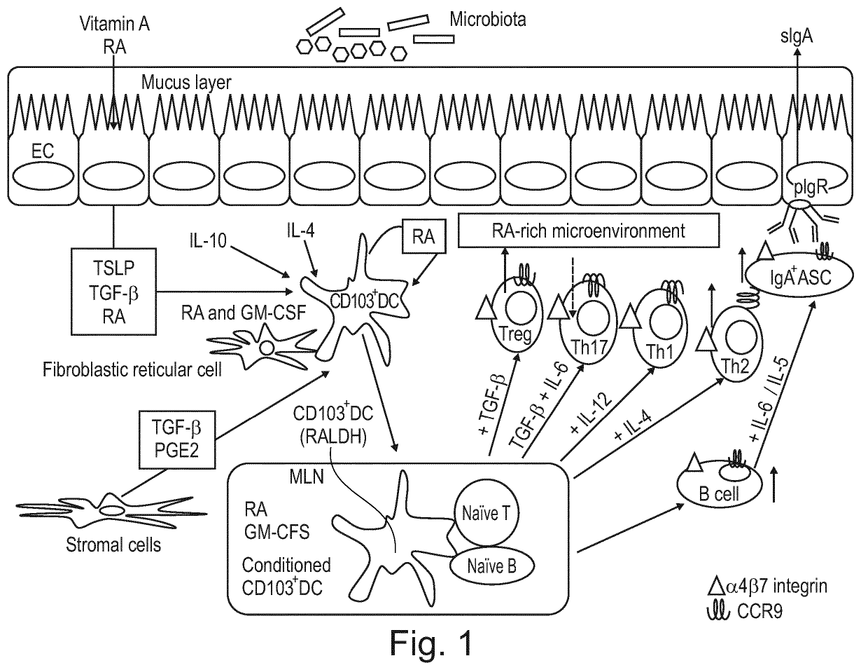

Vitamin A was already in the 1980's found to control the transcellular transport of the IgA dimers across the epithelial cells. During the following decades the impact of vitamin A interacting with several immune cells and stromal cells in the lamina propria (FIG. 1) was further explored.

One special characteristic of mucosal immune cells is their unique mucosal-imprinting phenotype, a property required in subsequent steps in the production and secretion of IgA antibody isotype (FIG. 1). This special property appears to require the presence of RA in the mucosal environment. The key finding on the influence of vitamin A (or RA) on the regulation of mucosal immune response was that RA has a central role in differentiation of DCs and that the mucosal DCs could metabolize retinol into retinoic acid.

Another important function of RA is to promote DC-dependent generation of IgA-antibody secreting cells from B cells and this process is enhanced by IgA-inducing cytokines like IL-5/IL-6. In fact, different lines of evidence from several animal models and human studies all agree that the synthesis of RA by lymphoid tissue DCs and other non-immune cells is needed to induce IgA expression in B cells. It is concluded from these studies that RA functions as a specific IgA isotype switching factor that facilitates the differentiation of IgA+antibody secreting cells and enhances IgA production in the presence of TGF-β. The effectiveness of this action is subjected to modulation by the presence of IL-5 or IL-6 in the microenvironment.

In a first aspect the present invention provides a method for vaccination, wherein a composition comprising an antigen, a TLR2 agonist and at least one pharmaceutically acceptable excipient is for pulmonal or intranasal administration, and wherein vitamin A is orally administered at least once within three days before or after the administration of said composition.

In one embodiment the method for vaccination comprises oral administration of vitamin D either before, at the same time or within 3 days from the administration of said composition.

In another embodiment the method for vaccination comprises oral administration of vitamin D in the period between one week before the administration of said composition and two days after the administration of said composition.

In another embodiment the method for vaccination includes said vitamin A to be orally administered at least once in the period between one day before the administration of said composition and two days after the administration of said composition.

In another embodiment of the method for vaccination said antigen is a protein or a multimer thereof, a peptide or a multimer thereof, an attenuated bacterium or an attenuated virus.

In an embodiment of the method for vaccination said antigen is attenuated SARS-Cov-2 or a component thereof.

In another embodiment of the method for vaccination said antigen is the spike protein from SARS-Cov-2 or a part thereof.

In yet another embodiment of the method for vaccination said TLR2 agonist is the compound of Formula (I):

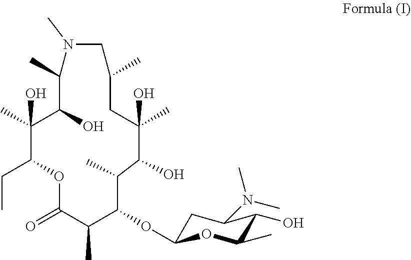

or a pharmaceutically acceptable salt thereof.

In yet another embodiment of the method for vaccination said TLR2 agonist is an analogue of the compound of Formula (I), wherein said analogue is a compound of Formula (Ia) or a pharmaceutically acceptable salt, hydrate, solvate, tautomer, enantiomer or diastereomer thereof:

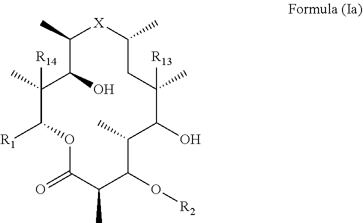

wherein X is selected from C═O, —NR₃CH₂—, —CH₂NR₃—, —NR₃(C═O)—, —(C═O)NR₃—, C═NOH, and —CH(OH)—, and R₂ is a sugar of Formula (II) or Formula (III):

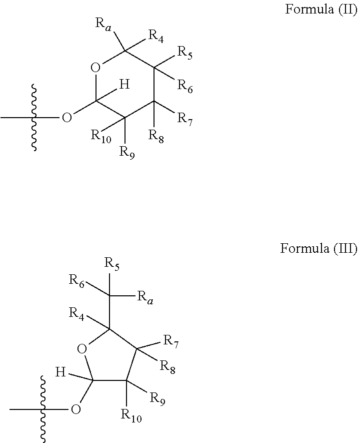

wherein R₁ is selected from an alkyl, heteroalkyl, cycloalkyl, aryl, and heteroaryl moiety, wherein alkyl moiety is selected from C₁-C₈ alkyl groups that are optionally branched,
wherein heteroalkyl moiety is selected from C₁-C₈ alkyl groups that are optionally branched or substituted and that optionally comprise one or more heteroatoms,
wherein cycloalkyl moiety is selected from a C₁-C₆ cyclic alkyl groups that are optionally substituted and that optionally comprise one or more heteroatoms,
wherein aryl moiety is selected from optionally substituted C aromatic rings,
wherein heteroaryl moiety is selected from optionally substituted C₁-C₈ aromatic rings comprising one or more heteroatoms,
wherein heteroatoms are selected from O, N, P, and S,
wherein substituents, independently, are selected from alkyl, OH, F, Cl, NH₂, NH-alkyl, NH-acyl, S-alkyl, S-acyl, O-alkyl, and O-acyl,
wherein acyl is selected from C₁-C₄ optionally branched acyl groups,
wherein R₃ is selected from H and Me,
wherein R₄ is selected from H and Me,
wherein Rₐ is selected from H and CR₂₁R₂₂R₂₃,
wherein R₂₁, R₂₂, R₂₃, and R₅, R₆, R₇, R₃, R₉, and R₁₀, independently, are selected from H, Me, NR₁₁R₁₂, NO₂, and OR₁₁,
wherein R₂₃ together with R₄ in Formula (II), R₄ together with R₅ in Formula (II), R₅ together with R₇ in Formula (II), and R₇ together with R₉ in Formula (II), independently, may be joined to represent a bond to leave a double bond between the carbon atoms that each group is connected to,
wherein R₂₁ together with R₂₂, R₅ together with R₆, R₇ together with R₃, or R₉ together with R₁₀ may be replaced with a carbonyl,
wherein R₁₁ and R₁₂, independently, are selected from H and alkyl,
wherein R₁₃ is selected from H, OH, and OCH₃,
wherein R₁₄ is selected from H and OH,
and wherein one of R₅, R₆, R₇, R₃, R₉ or R₁₀ is selected from NR₁₁R₁₂ and NO₂,
with the proviso that when R₁ is Et, R₂ is a sugar of Formula (II), R₁₃ is H or OH, R₁₄ is H or OH, Rₐ is H, R₄ is Me, R₅ is H, R₅ is OH, R₇ is H, R₈ is NR₁₁R₁₂, R₉ is H, and R₁₀ is H, X may not be C═O.
with the proviso that when R₁ is Et, R₂ is a sugar of Formula (II), R₁₃ is H or OH, R₁₄ is H or OH, Rₐ is H, R₄ is Me, R₅ is OH, R₆ is H, R₇ is OH, R₃ is Me, R₉ is H, and R₁₀ is H, X may not be C═O. with the proviso that when R₁ is Et, R₂ is a sugar of Formula (II), R₁₃ is H or OH, R₁₄ is
H or OH, Rₐ is H, R₄ is Me, R₅ is OH, R₅ is H, R₇ is H, R₈ is NR₁₁R₁₂, R₉ is H, and R₁₀ is OH, X may not be C═O.
   1. In yet another embodiment of the method for vaccination said TLR2 agonist is selected from:

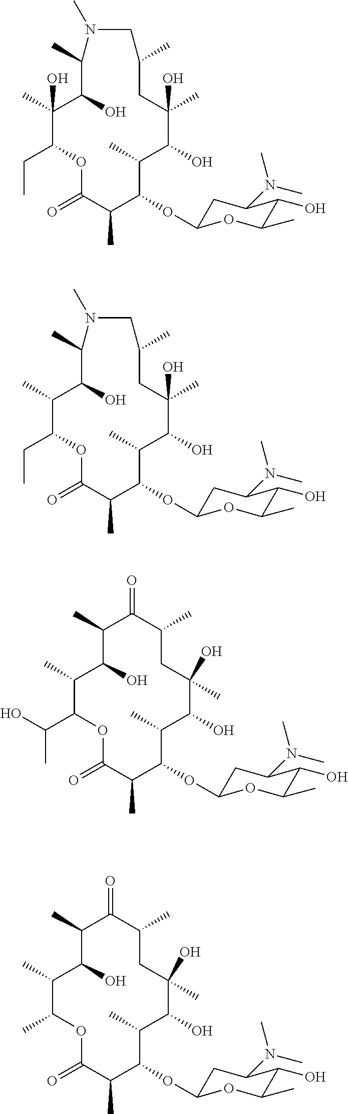 [SMILES: CC[C@H]1OC(=O)[C@H](C)[C@@H](OC2CC(N(C)C)C(O)C(C)O2)[C@H](C)[C@@H](O)[C@](C)(O)C[C@@H](C)CN(C)[C@H](C)[C@@H](O)[C@]1(C)O ; CC[C@H]1OC(=O)[C@H](C)[C@@H](OC2CC(N(C)C)C(O)C(C)O2)[C@H](C)[C@@H](O)[C@](C)(O)C[C@@H](C)CN(C)[C@H](C)[C@@H](O)[C@H]1C ; CC(O)C1OC(=O)[C@H](C)[C@@H](OC2CC(N(C)C)C(O)C(C)O2)[C@H](C)[C@@H](O)[C@](C)(O)C[C@@H](C)C(=O)[C@H](C)[C@@H](O)[C@H]1C ; CC1OC(O[C@H]2[C@H](C)[C@@H](O)[C@](C)(O)C[C@@H](C)C(=O)[C@H](C)[C@@H](O)[C@@H](C)[C@@H](C)OC(=O)[C@@H]2C)CC(N(C)C)C1O]

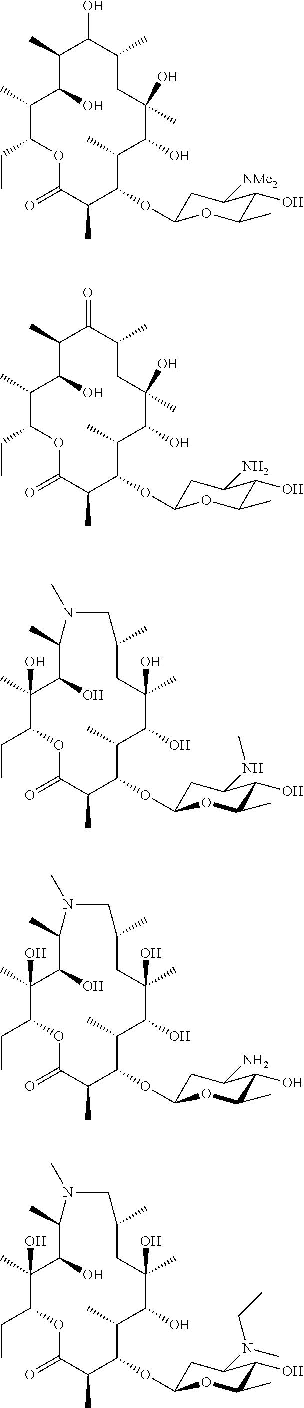

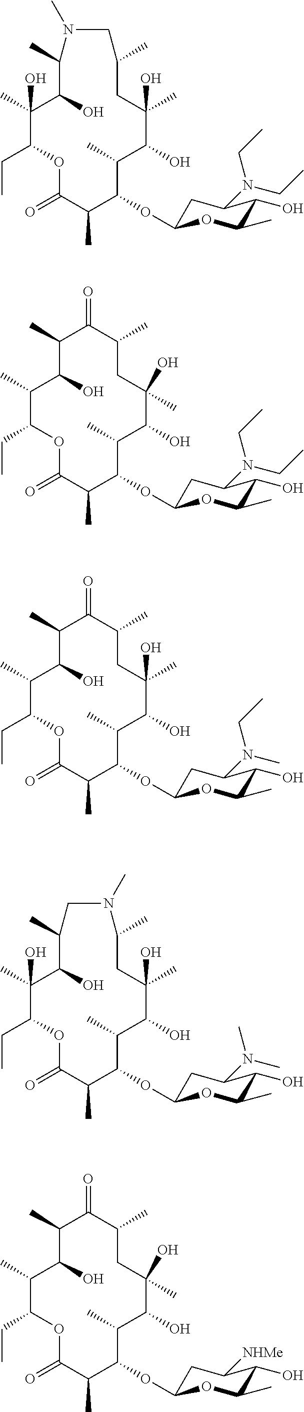

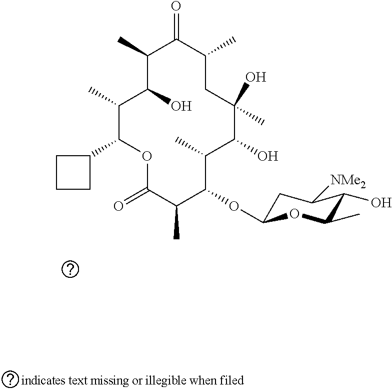

In a second aspect the present invention provides a vaccine kit comprising:
a composition comprising an antigen, a TLR2 agonist and at least one pharmaceutically acceptable excipient, and
a label informing that said composition is to be used for vaccination by co-administration of vitamin A.

In one embodiment the vaccine kit comprises said label further informing that said composition is to be used for vaccination by co-administration of vitamin D.

In yet another embodiment the vaccine kit comprises said label informing that vitamin D is administered orally.

In another embodiment of the vaccine kit said composition is for pulmonary or intranasal administration.

In another embodiment of the vaccine kit said label informs that vitamin A is administered orally.

In a third aspect the present invention provides a vaccine kit comprising
a first composition comprising an antigen, a TLR2 agonist and at least one pharmaceutically acceptable excipient, and
a second composition comprising vitamin A.

In one embodiment the vaccine kit has said second composition to comprise vitamin D.

In another embodiment the vaccine kit comprises a third composition comprising vitamin D. In another embodiment said third composition is for oral administration.

In another embodiment the vaccine kit has said first composition adapted for pulmonary or intranasal administration.

In another embodiment the vaccine kit has said second composition adapted for oral administration.

In an embodiment the vaccine kit has said antigen being a protein or a multimer thereof, a peptide or a multimer thereof, an attenuated bacterium or an attenuated virus.

In another embodiment the vaccine kit has said antigen being attenuated SARS-Cov-2 or a component thereof. In yet another embodiment the vaccine kit has said antigen being the spike protein from SARS-Cov-2 or a part thereof.

In another embodiment the vaccine kit has said TLR2 agonist being the compound of Formula (I):

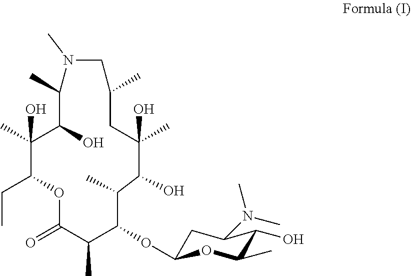

or a pharmaceutically acceptable salt thereof.

In another embodiment the vaccine kit has said TLR2 agonist being an analogue of the compound of Formula (I), wherein said analogue is a compound of Formula (Ia) or a pharmaceutically acceptable salt, hydrate, solvate, tautomer, enantiomer or diastereomer thereof:

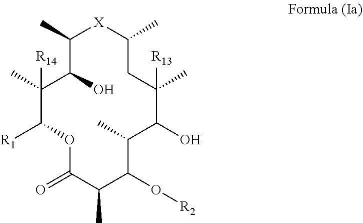

wherein X is selected from C═O, —NR₃CH₂—, —CH₂NR₃—, —NR₃(C═O)—, —(C═O)NR₃—, C═NOH, and —CH(OH)—, and R₂ is a sugar of Formula (II) or Formula (III):

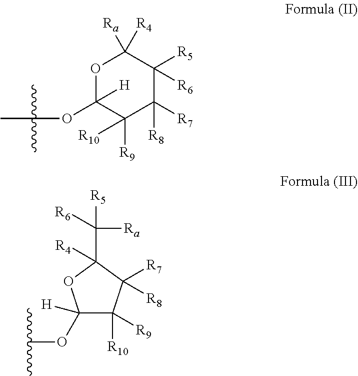

wherein R₁ is selected from an alkyl, heteroalkyl, cycloalkyl, aryl, and heteroaryl moiety, wherein alkyl moiety is selected from C₁-C₆ alkyl groups that are optionally branched,
wherein heteroalkyl moiety is selected from C₁-C₆ alkyl groups that are optionally branched or substituted and that optionally comprise one or more heteroatoms,
wherein cycloalkyl moiety is selected from a C₁—C cyclic alkyl groups that are optionally substituted and that optionally comprise one or more heteroatoms,
wherein aryl moiety is selected from optionally substituted C aromatic rings,
wherein heteroaryl moiety is selected from optionally substituted C₁-C₅ aromatic rings comprising one or more heteroatoms,
wherein heteroatoms are selected from O, N, P, and S,
wherein substituents, independently, are selected from alkyl, OH, F, Cl, NH₂, NH-alkyl, NH-acyl, S-alkyl, S-acyl, O-alkyl, and O-acyl,
wherein acyl is selected from C₁-C₄ optionally branched acyl groups,
wherein R₃ is selected from H and Me,
wherein R₄ is selected from H and Me,
wherein Rₐ is selected from H and CR₂₁R₂₂R₂₃,
wherein R₂₁, R₂₂, R₂₃, and R₅, R₆, R₇, R₈, R₉, and R₁₀, independently, are selected from H, Me, NR₁₁R₁₂, NO₂, and OR₁₁,
wherein R₂₃ together with R₄ in Formula (II), R₄ together with R₅ in Formula (II), R₅ together with R₇ in Formula (II), and R₇ together with R₉ in Formula (II), independently, may be joined to represent a bond to leave a double bond between the carbon atoms that each group is connected to,
wherein R₂₁ together with R₂₂, R₅ together with R₆, R₇ together with R₃, or R₉ together with R₁₀ may be replaced with a carbonyl,
wherein R₁₁ and R₁₂, independently, are selected from H and alkyl,
wherein R₁₃ is selected from H, OH, and OCH₃,
wherein R₁₄ is selected from H and OH,
and wherein one of R₅, R₆, R₇, R₃, R₉ or R₁₀ is selected from NR₁₁R₁₂ and NO₂,
with the proviso that when R₁ is Et, R₂ is a sugar of Formula (II), R₁₃ is H or OH, R₁₄ is H or OH, Ra is H, R₄ is Me, R₅ is H, R₅ is OH, R₇ is H, R₈ is NR₁₁R₁₂, R₉ is H, and R₁₀ is H, X may not be C═O.
with the proviso that when R₁ is Et, R₂ is a sugar of Formula (II), R₁₃ is H or OH, R₁₄ is H or OH, Rₐ is H, R₄ is Me, R₅ is OH, R₆ is H, R₇ is OH, R₃ is Me, R₉ is H, and R₁₀ is H, X may not be C═O.
with the proviso that when R₁ is Et, R₂ is a sugar of Formula (II), R₁₃ is H or OH, R₁₄ is H or OH, Rₐ is H, R₄ is Me, R₅ is OH, R₅ is H, R₇ is H, R₈ is NR₁₁R₁₂, R₉ is H, and R₁₀ is OH, X may not be C═O.

In yet another embodiment the vaccine kit has said TLR2 agonist being selected from:

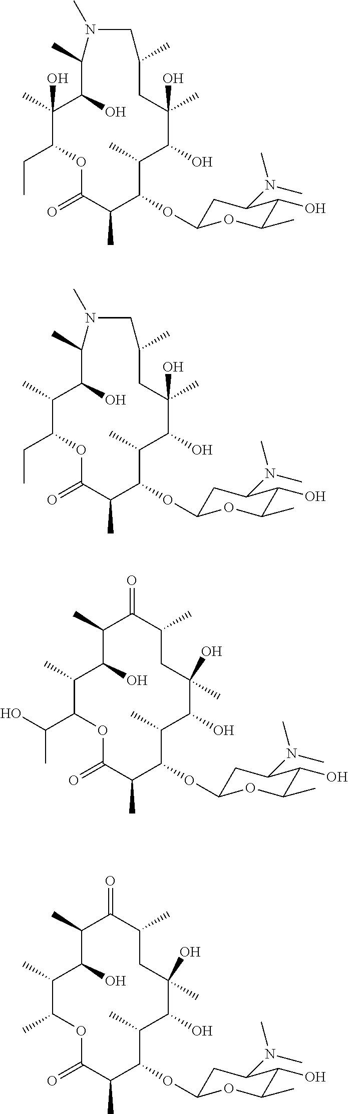 [SMILES: CC[C@H]1OC(=O)[C@H](C)[C@@H](OC2CC(N(C)C)C(O)C(C)O2)[C@H](C)[C@@H](O)[C@](C)(O)C[C@@H](C)CN(C)[C@H](C)[C@@H](O)[C@]1(C)O ; CC[C@H]1OC(=O)[C@H](C)[C@@H](OC2CC(N(C)C)C(O)C(C)O2)[C@H](C)[C@@H](O)[C@](C)(O)C[C@@H](C)CN(C)[C@H](C)[C@@H](O)[C@H]1C ; CC(O)C1OC(=O)[C@H](C)[C@@H](OC2CC(N(C)C)C(O)C(C)O2)[C@H](C)[C@@H](O)[C@](C)(O)C[C@@H](C)C(=O)[C@H](C)[C@@H](O)[C@H]1C ; CC1OC(O[C@H]2[C@H](C)[C@@H](O)[C@](C)(O)C[C@@H](C)C(=O)[C@H](C)[C@@H](O)[C@@H](C)[C@@H](C)OC(=O)[C@@H]2C)CC(N(C)C)C1O]

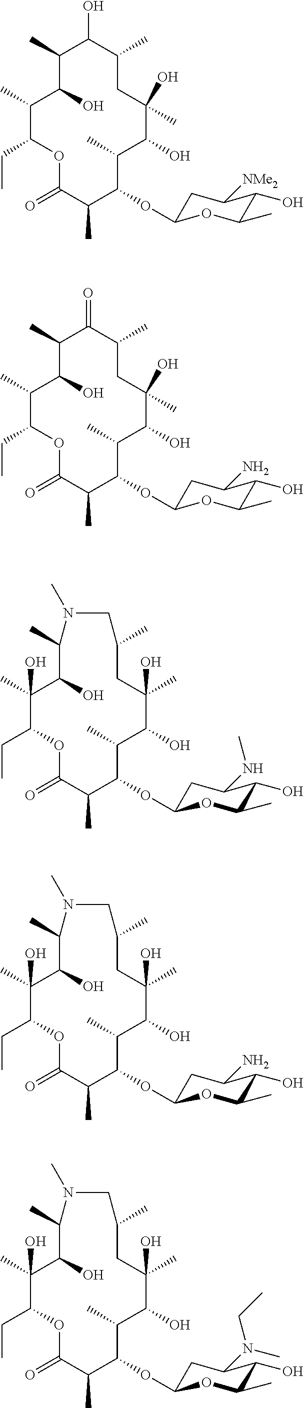

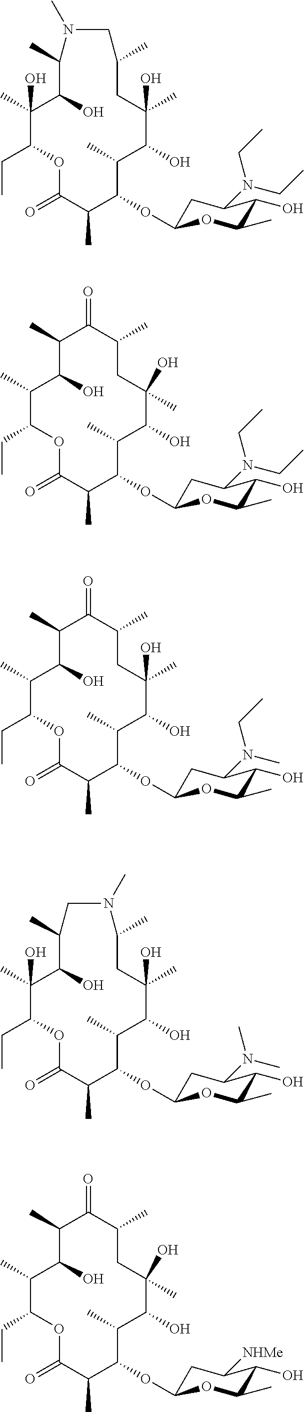

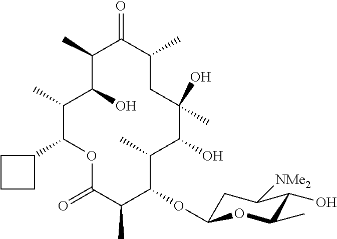

General Chemistry Methods

The skilled person will recognise that the TLR2 agonists for use in the invention may be prepared, in known manner, in a variety of ways. The routes below are merely illustra-tive of some methods that can be employed for the synthesis of compounds of formula (I).

In one general route to prepare e.g. the compound of Formula (I), erythromycin A is subjected to semisynthetic manipulation to generate azithromycin. Methods for this transformation are known (U.S. Pat. Nos. 3,478,014; 4,328,334; 4,474,768, Glansdorp et al., 2008, though variants on these routes or other routes may be used to the same purpose. The mycarose/cladinose and/or desosamine are removed by further chemi-cal methods, such as glycoside cleavage. Briefly, in one method the sugars may be removed by treatment with acid. In order to facilitate removal of the amino sugar it is first necessary to oxidise the dimethylamine to form an N-oxide which is then removed by pyrolysis. The resultant 5-0 sugar, and 3-0 sugar, can then be removed by acidic deg-radation. A suitable method is taught by LeMahieu (1974) and Djokic, S., et al., 1988.

Finally, the compound is biotransformed using a bacterial strain which adds the amino sugar.

General Use of the Vaccines of the Invention

The vaccinations methods and the vaccine compositions of the invention disclosed herein may be used to provide individuals with immunity against viral agents, and in particular against respiratory viruses.

Pharmaceutical Compositions for Use in the Method for Vaccination of the Invention

The present invention also provides vaccination kits comprising a pharmaceutical composition comprising the antigen and a TLR2 agonist together with at least one pharmaceutically acceptable excipient. The present invention also relates to pulmonal or intranasal compositions comprising the antigen and a TLR2 agonist together with at least one pharmaceutically acceptable excipient Pharmaceutical compositions for pulmonary administration may be liquid or solid form-lations for administration as vapour or aerosols. Aerosols may be delivered by jet or mesh nebulizers, where the mesh nebulizers have higher aerosolization efficiencies and more rapid administration compared to the traditional jet nebulizers. Solid formulations for pulmonary administration may be delivered by dry powder inhalers.

The vaccination method may consist of a single administration or a plurality of administrations over a period of time. In particular, the oral administration of vitamin A may consist of a plurality of administrations.

The formulations may conveniently be presented in a suitable dosage form including a unit dosage form and may be prepared by any of the methods well known in the art of pharmacy. Such methods include the step of bringing into association the active ingredient (antigen) and the TLR2 agonist with the at least one excipient. In general, the formulations are prepared by uniformly and intimately bringing into association the active ingredient with liquid carriers or finely divided solid carriers or both.

Depending upon the particular vaccination and the individual to be vaccinated, as well as the route of administration, the compositions may be administered at varying doses and/or frequencies.

The pharmaceutical compositions must be stable under the conditions of manufacture and storage; thus, if necessary they should be preserved against the contaminating action of microorganisms such as bacteria and fungi. In case of liquid formulations such as solutions, dispersion and suspensions, the carrier can be a solvent or dispersion medium containing, for example, water, ethanol, polyol (e.g. glycerol, propylene glycol and liquid polyethylene glycol), vegetable oils, and suitable mixtures thereof. In case of solid formulations, dry powder formulations are usually prepared by mixing the mi-cronized active particles with larger carrier particles such as lactose or mannitol. The aerosolization efficiency of a powder is highly dependent on the carrier characteristics, such as particle size distribution, shape and surface properties.

The compositions for use in the vaccination methods of the invention comprises at least one pharmaceutically acceptable excipient, such as carriers, solvents, propel-lants, pH-adjusting agents, stabilizing agents, surfactants, solubilizers, dispersing agents, preservatives etc.

It should be understood that in addition to the ingredients particularly mentioned above the formulations of this invention may include other agents conventional in the art hav-ing regard to the type of formulation in question. A person skilled in the art will know how to choose a suitable formulation and how to prepare it (see eg Remington's Pharmaceutical Sciences 18 Ed. or later). A person skilled in the art will also know how to choose a suitable administration route and dosage.

The pharmaceutically acceptable salts of the TLR2 agonist include conventional salts formed from pharmaceutically acceptable inorganic or organic acids or bases as well as quaternary ammonium acid addition salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malo-nic, hydroxymaleic, phenylacetic, glutamic, benzoic, salicylic, toluenesulfonic, me-thanesulfonic, naphthalene-2-sulfonic, benzenesulfonic hydroxynaphthoic, hydroiodic, malic, steroic, tannic and the like. Other acids such as oxalic, while not in themselves pharmaceutically acceptable, may be useful in the preparation of salts useful as inter-mediates in obtaining the compounds of the invention and their pharmaceutically acceptable salts. More specific examples of suitable basic salts include sodium, lithium, potassium, magnesium, aluminium, calcium, zinc, N,N′-dibenzylethylenediamine, chlo-roprocaine, choline, diethanolamine, ethylenediamine, N-methylglucamine and pro-caine salts.

The following list of non-limiting embodiments further illustrate the invention:
1. A method for vaccination, wherein a composition comprising an antigen, a TLR2 agonist and at least one pharmaceutically acceptable excipient is for pulmonal or intranasal administration, and wherein vitamin A is orally administered at least once within three days before or after the administration of said composition.
2. The method for vaccination according to embodiment 1, wherein said antigen is a protein or a multimer thereof, a peptide or a multimer thereof, an attenuated bacterium or an attenuated virus.
3. The method for vaccination according to any of embodiments 1-2, wherein said TLR2 agonist is the compound of Formula (I):

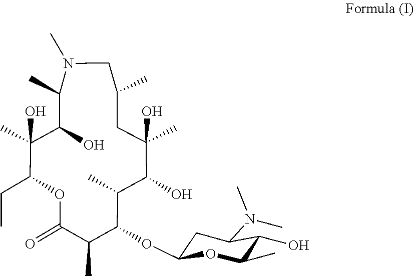

or a pharmaceutically acceptable salt thereof.
4. The method for vaccination according to any of embodiments 1-2, wherein said TLR2 agonist is an analogue of the compound of Formula (I), wherein said analogue is a compound of Formula (Ia) or a pharmaceutically acceptable salt, hydrate, solvate, tautomer, enantiomer or diastereomer thereof:

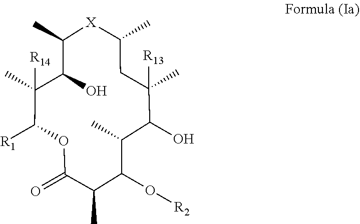

wherein X is selected from C═O, —NR₃CH₂—, —CH₂NR₃—, —NR₃(C═O)—, —(C═O)NR₃—, C═NOH, and —CH(OH)—, and R₂ is a sugar of Formula (II) or Formula (III):

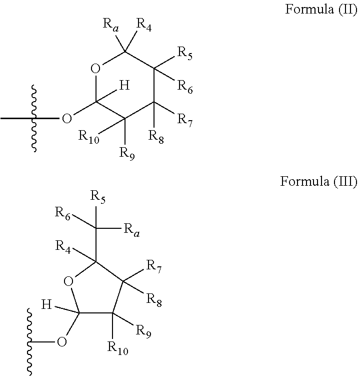

wherein R₁ is selected from an alkyl, heteroalkyl, cycloalkyl, aryl, and heteroaryl moiety,
wherein alkyl moiety is selected from C₁-C₆ alkyl groups that are optionally branched,
wherein heteroalkyl moiety is selected from C₁-C₈ alkyl groups that are optionally branched or substituted and that optionally comprise one or more heteroatoms,
wherein cycloalkyl moiety is selected from a C₁-C₆ cyclic alkyl groups that are optionally substituted and that optionally comprise one or more heteroatoms,
wherein aryl moiety is selected from optionally substituted C₆ aromatic rings, wherein heteroaryl moiety is selected from optionally substituted C₁-C₅ aromatic rings comprising one or more heteroatoms,
wherein heteroatoms are selected from O, N, P, and S,
wherein substituents, independently, are selected from alkyl, OH, F, Cl, NH₂, NH-alkyl, NH-acyl, S-alkyl, S-acyl, O-alkyl, and O-acyl,
wherein acyl is selected from C₁-C₄ optionally branched acyl groups,
wherein R₃ is selected from H and Me,
wherein R₄ is selected from H and Me,
wherein Rₐ is selected from H and CR₂₁R₂₂R₂₃,
wherein R₂₁, R₂₂, R₂₃, and R₅, R₆, R₇, R₃, R₉, and R₁₀, independently, are selected from H, Me, NR₁₁R₁₂, NO₂, and OR₁₁,
wherein R₂₃ together with R₄ in Formula (II), R₄ together with R₅ in Formula (II), R₅ together with R₇ in Formula (II), and R₇ together with R₉ in Formula (II), independently, may be joined to represent a bond to leave a double bond between the carbon atoms that each group is connected to,
wherein R₂₁ together with R₂₂, R₅ together with R₆, R₇ together with R₃, or R₉ together with R₁₀ may be replaced with a carbonyl,
wherein R₁₁ and R₁₂, independently, are selected from H and alkyl,
wherein R₁₃ is selected from H, OH, and OCH₃,
wherein R₁₄ is selected from H and OH,
and wherein one of R₅, R₆, R₇, R₃, R₉ or R₁₀ is selected from NR₁₁R₁₂ and NO₂,
with the proviso that when R₁ is Et, R₂ is a sugar of Formula (II), R₁₃ is H or OH, R₁₄ is H or OH, Ra is H, R₄ is Me, R₅ is H, R₅ is OH, R₇ is H, R₈ is NR₁₁R₁₂, R₉ is H, and R₁₀ is H, X may not be C═O.
with the proviso that when R₁ is Et, R₂ is a sugar of Formula (II), R₁₃ is H or OH, R₁₄ is H or OH, Ra is H, R₄ is Me, R₅ is OH, R₆ is H, R₇ is OH, R₈ is Me, R₉ is H, and R₁₀ is H, X may not be C═O.
with the proviso that when R₁ is Et, R₂ is a sugar of Formula (II), R₁₃ is H or OH, R₁₄ is H or OH, Ra is H, R₄ is Me, R₅ is OH, R₅ is H, R₇ is H, R₈ is NR₁₁R₁₂, R₉ is H, and R₁₀ is OH, X may not be C═O.
5. The method for vaccination according to embodiment 4, wherein X is selected from —NR₃CH₂— and —CH₂NR₃ and R₂ is Formula (II):

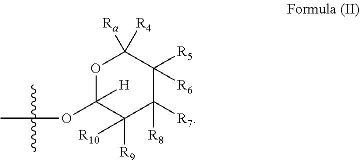

6. The method according to any of embodiments 4-5, wherein R₁ is methyl or ethyl.
7. The method according to any of embodiments 4-6, wherein one of R₅, R₆, R₇, or R₈, is NR₁₁R₁₂.
8. The method according to any of embodiments 4-7, wherein R₂₁, R₂₂, R₂₃, and R₅, R₆, R₇, R₈, R₉, and R₁₀, independently, are selected from H, Me, NR₁₁R₁₂, and OR₁₁,
9. The method according to any of embodiments 4-8, wherein R₁₃ and R₁₄ are OH
10. The method according to any of embodiments 4-9, wherein X is selected from —NR₃CH₂— and —CH₂NR₃ and R₂ is Formula (II):

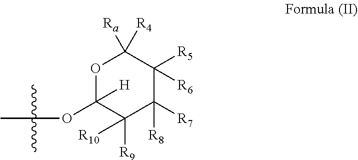

and
wherein R₁ is methyl or ethyl,
wherein R₃ is selected from H and Me,
wherein R₄ is H,
wherein Ra is —CR₂₁R₂₂R₂₃,
wherein R₂₁, R₂₂, R₂₃, and R₅, R₆, R₇, R₈, R₉, and R₁₀, independently, are selected from H, Me, NR₁₁R₁₂, NO₂, and OR₁₁,
wherein R₁₁ and R₁₂, independently, are selected from H and alkyl, wherein alkyl moiety is selected from C₁-C₈ alkyl groups that are optionally branched,
wherein R₁₃ is selected from H, OH, and OCH₃,
wherein R₁₄ is selected from H and OH,
and wherein one of R₅, R₆, R₇, R₈, R₉ or R₁₀ is NR₁₁R₁₂.
11. The method according to any of embodiments 4-10, wherein R₂ is a sugar according to formula II, wherein Rₐ is H, R₄ is Me, R₅ is H, R₆ is OH, R₇ is H, R₈ is NR₁₁R₁₂, R₉ is H and R₁₀ is H.
12. The method according to any of embodiments 4-11, wherein R₁₁ and R₁₂ independently are selected from H, Me, and Et.
13. The method according to any of embodiments 4-12, wherein X is —NR₃CH₂—.
14. The method according to any of embodiments 4-13, wherein R₁ is Et.
15. The method according to any of embodiments 4-14, wherein said TLR2 agonist is selected from:

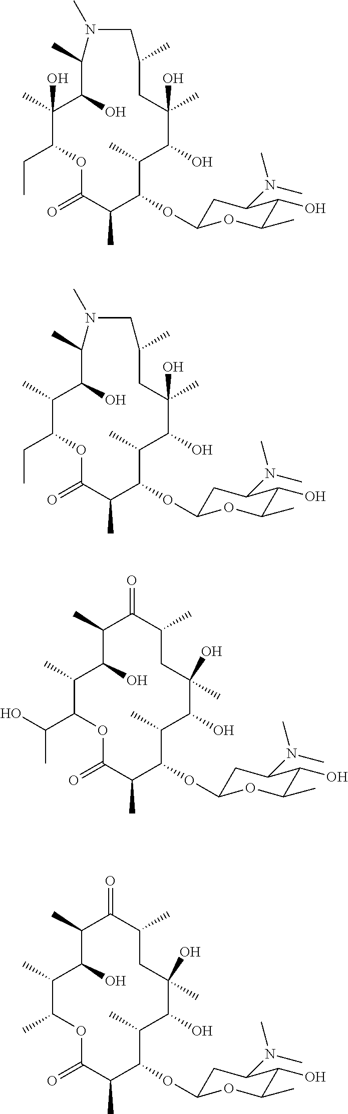 [SMILES: CC[C@H]1OC(=O)[C@H](C)[C@@H](OC2CC(N(C)C)C(O)C(C)O2)[C@H](C)[C@@H](O)[C@](C)(O)C[C@@H](C)CN(C)[C@H](C)[C@@H](O)[C@]1(C)O ; CC[C@H]1OC(=O)[C@H](C)[C@@H](OC2CC(N(C)C)C(O)C(C)O2)[C@H](C)[C@@H](O)[C@](C)(O)C[C@@H](C)CN(C)[C@H](C)[C@@H](O)[C@H]1C ; CC(O)C1OC(=O)[C@H](C)[C@@H](OC2CC(N(C)C)C(O)C(C)O2)[C@H](C)[C@@H](O)[C@](C)(O)C[C@@H](C)C(=O)[C@H](C)[C@@H](O)[C@H]1C ; CC1OC(O[C@H]2[C@H](C)[C@@H](O)[C@](C)(O)C[C@@H](C)C(=O)[C@H](C)[C@@H](O)[C@@H](C)[C@@H](C)OC(=O)[C@@H]2C)CC(N(C)C)C1O]

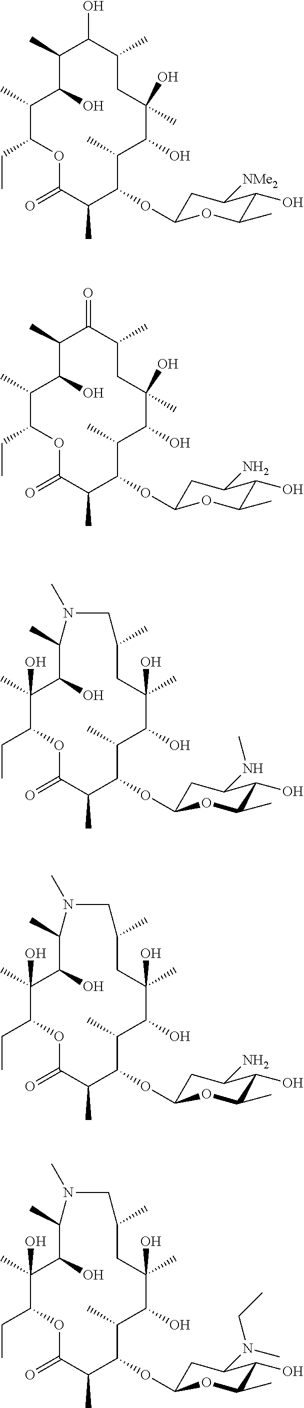

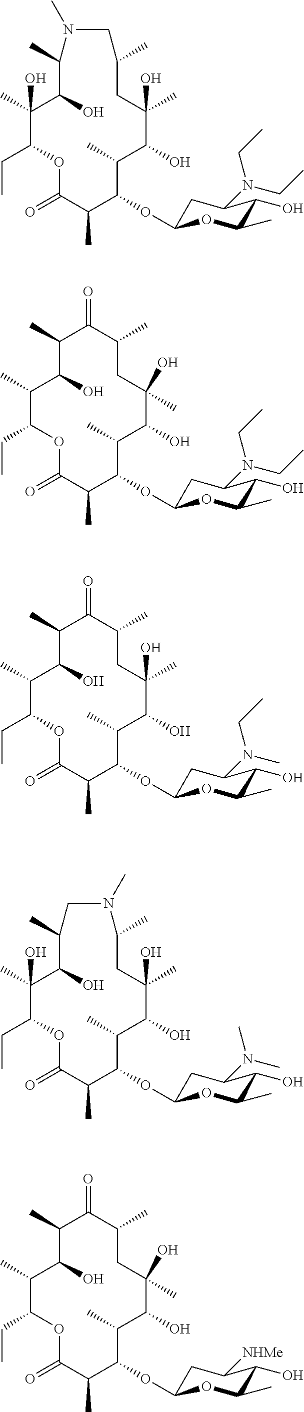

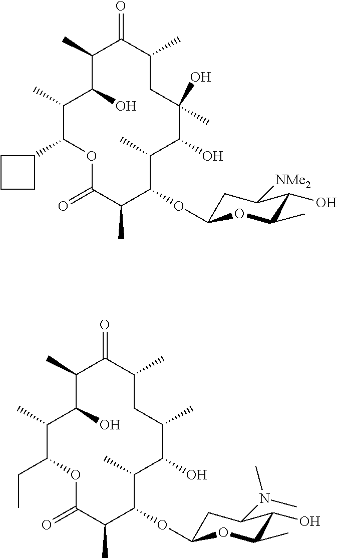

16. The method for vaccination according to any of embodiments 1-15, wherein vitamin D is administered orally either before, at the same time or within 3 days from the administration of said composition.
17. The method for vaccination according to embodiment 16, wherein vitamin D is administered orally in the period between one week before the administration of said composition and two days after the administration of said composition.
18. The method for vaccination according to any of embodiments 1-17, wherein said antigen is attenuated SARS-Cov-2 or a component thereof.
19. The method for vaccination according to any of embodiments 1-18, wherein said antigen is the spike protein from SARS-Cov-2 or a part thereof.
20. The method for vaccination according to any of embodiments 1-19, wherein said vitamin A is orally administered at least once in the period between one day before the administration of said composition and two days after the administration of said composition.
21. The method for vaccination according to any of embodiments 1-20, wherein said composition comprises poly 1:C.
22. A vaccine kit comprising:
   a composition comprising an antigen, a TLR2 agonist and at least one pharmaceutically acceptable excipient, and
   a label informing that said composition is to be used for vaccination by co-administration of vitamin A.
23. The vaccine kit according to embodiment 22, wherein said antigen is a protein or a multimer thereof, a peptide or a multimer thereof, an attenuated bacterium or an attenuated virus.
24. The vaccine kit according to any of embodiments 22-23, wherein said TLR2 agonist is a compound of Formula (I):

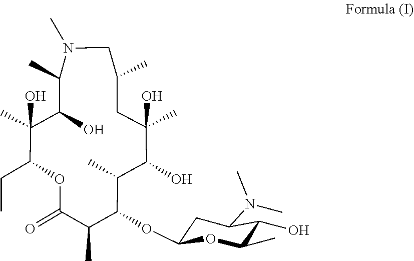

or a pharmaceutically acceptable salt thereof.
25. The vaccine kit according to any of embodiments 22-23, wherein said TLR2 agonist is an analogue of the compound of Formula (I), wherein said analogue is a compound of Formula (Ia) or a pharmaceutically acceptable salt, hydrate, solvate, tautomer, enantiomer or diastereomer thereof:

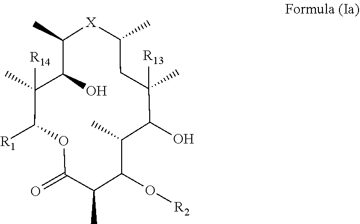

wherein X is selected from C═O, —NR₃CH₂—, —CH₂NR₃—, —NR₃(C═O)—, —(C═O)NR₃, —C═NOH, and —CH(OH)—, and R₂ is a sugar of Formula (II) or Formula (III):

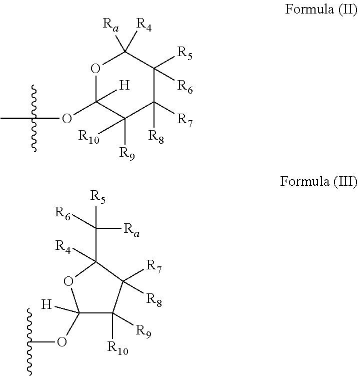

wherein R₁ is selected from an alkyl, heteroalkyl, cycloalkyl, aryl, and heteroaryl moiety,
wherein alkyl moiety is selected from C₁-C₆ alkyl groups that are optionally branched,
wherein heteroalkyl moiety is selected from C₁-C₆ alkyl groups that are optionally branched or substituted and that optionally comprise one or more heteroatoms,
wherein cycloalkyl moiety is selected from a C₁-C₆ cyclic alkyl groups that are optionally substituted and that optionally comprise one or more heteroatoms,
wherein aryl moiety is selected from optionally substituted C₆ aromatic rings,
wherein heteroaryl moiety is selected from optionally substituted C₁-C₈ aromatic rings comprising one or more heteroatoms,
wherein heteroatoms are selected from O, N, P, and S,
wherein substituents, independently, are selected from alkyl, OH, F, Cl, NH₂, NH-alkyl, NH-acyl, S-alkyl, S-acyl, O-alkyl, and O-acyl,
wherein acyl is selected from C₁-C₄ optionally branched acyl groups,
wherein R₃ is selected from H and Me,
wherein R₄ is selected from H and Me,
wherein Rₐ is selected from H and CR₂₁R₂₂R₂₃,
wherein R₂₁, R₂₂, R₂₃, and R₅, R₆, R₇, R₃, R₉, and R₁₀, independently, are selected from H, Me, NR₁₁R₁₂, NO₂, and OR₁₁,
wherein R₂₃ together with R₄ in Formula (II), R₄ together with R₅ in Formula (II), R₅ together with R₇ in Formula (II), and R₇ together with R₉ in Formula (II), independently, may be joined to represent a bond to leave a double bond between the carbon atoms that each group is connected to,
wherein R₂₁ together with R₂₂, R₅ together with R₆, R₇ together with R₃, or R₉ together with R₁₀ may be replaced with a carbonyl,
wherein R₁₁ and R₁₂, independently, are selected from H and alkyl,
wherein R₁₃ is selected from H, OH, and OCH₃,
wherein R₁₄ is selected from H and OH,
and wherein one of R₅, R₆, R₇, R₃, R₉ or R₁₀ is selected from NR₁₁R₁₂ and NO₂,
with the proviso that when R₁ is Et, R₂ is a sugar of Formula (II), R₁₃ is H or OH, R₁₄ is H or OH, Ra is H, R₄ is Me, R₅ is H, R₆ is OH, R₇ is H, R₈ is NR₁₁R₁₂, R₉ is H, and R₁₀ is H, X may not be C═O.
with the proviso that when R₁ is Et, R₂ is a sugar of Formula (II), R₁₃ is H or OH, R₁₄ is H or OH, Rₐ is H, R₄ is Me, R₅ is OH, R₆ is H, R₇ is OH, R₃ is Me, R₉ is H, and R₁₀ is H, X may not be C═O.
with the proviso that when R₁ is Et, R₂ is a sugar of Formula (II), R₁₃ is H or OH, R₁₄ is H or OH, Ra is H, R₄ is Me, R₅ is OH, R₆ is H, R₇ is H, R₈ is NR₁₁R₁₂, R₉ is H, and R₁₀ is OH, X may not be C═O.
26. The vaccine kit according to embodiment 25, wherein said TLR2 agonist is selected from:

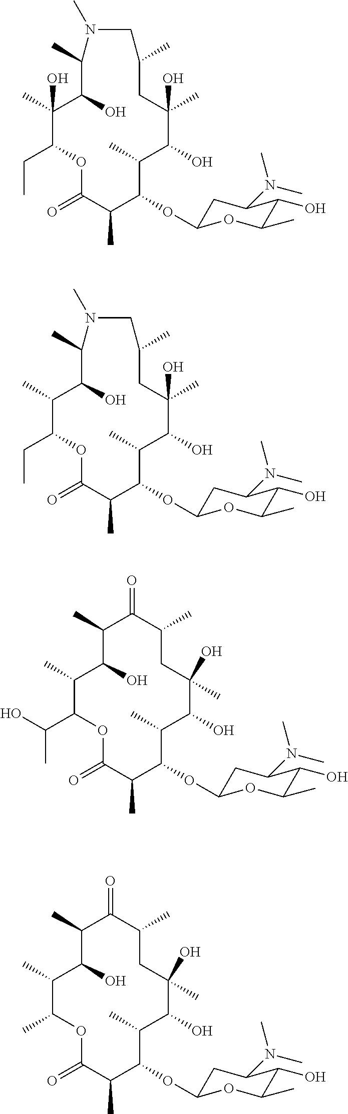 [SMILES: CC[C@H]1OC(=O)[C@H](C)[C@@H](OC2CC(N(C)C)C(O)C(C)O2)[C@H](C)[C@@H](O)[C@](C)(O)C[C@@H](C)CN(C)[C@H](C)[C@@H](O)[C@]1(C)O ; CC[C@H]1OC(=O)[C@H](C)[C@@H](OC2CC(N(C)C)C(O)C(C)O2)[C@H](C)[C@@H](O)[C@](C)(O)C[C@@H](C)CN(C)[C@H](C)[C@@H](O)[C@H]1C ; CC(O)C1OC(=O)[C@H](C)[C@@H](OC2CC(N(C)C)C(O)C(C)O2)[C@H](C)[C@@H](O)[C@](C)(O)C[C@@H](C)C(=O)[C@H](C)[C@@H](O)[C@H]1C ; CC1OC(O[C@H]2[C@H](C)[C@@H](O)[C@](C)(O)C[C@@H](C)C(=O)[C@H](C)[C@@H](O)[C@@H](C)[C@@H](C)OC(=O)[C@@H]2C)CC(N(C)C)C1O]

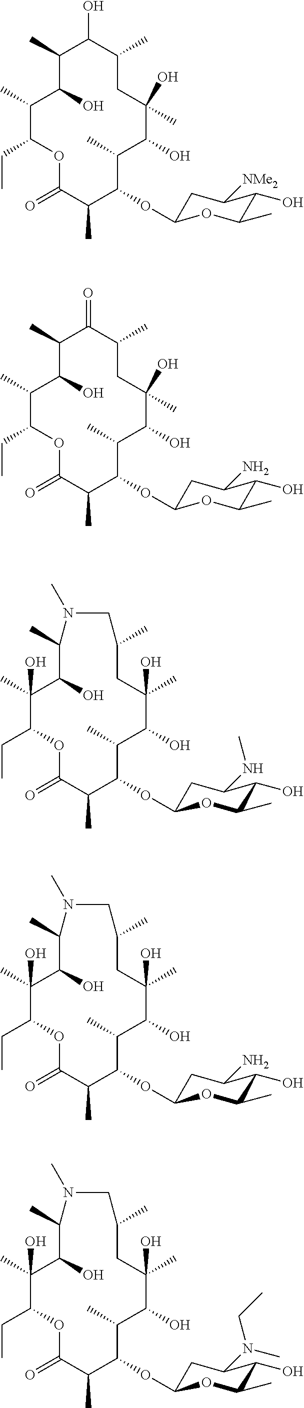

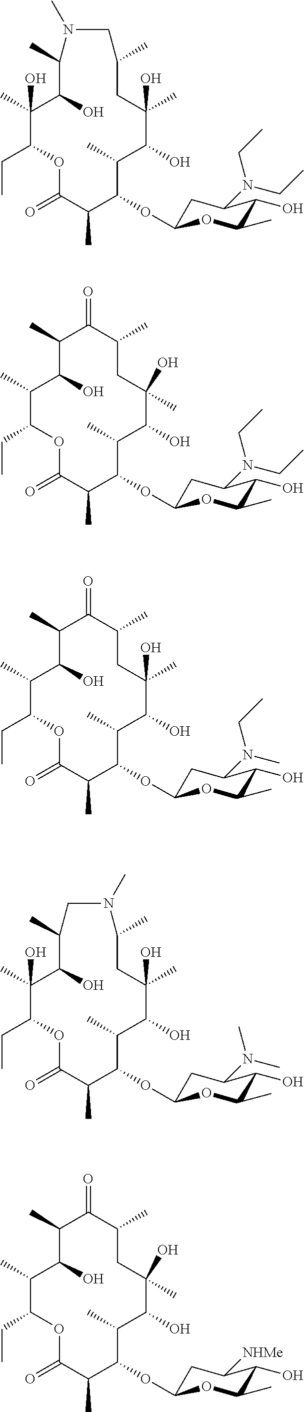

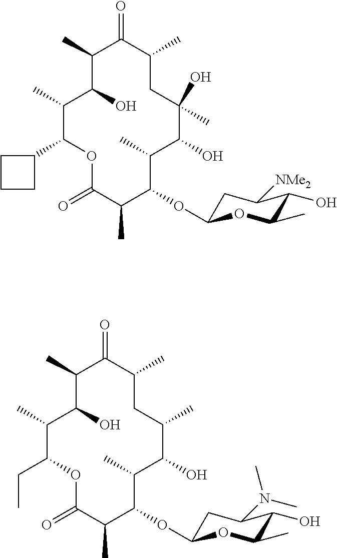

27. The vaccine kit according to any of embodiments 22-26, wherein said label further informs that said composition is to be used for vaccination by co-administration of vitamin D.
28. The vaccine kit according to any of embodiments 22-27, wherein said composition is for pulmonary or intranasal administration.
29. The vaccine kit according to any of embodiments 22-28, wherein said label informs that vitamin A is administered orally.
30. The vaccine kit according to any of embodiments 27-29, wherein said label informs that vitamin D is administered orally.
31. The vaccine kit according to any of embodiments 22-30, wherein said antigen is attenuated SARS-Cov-2 or a component thereof.
32. The vaccine kit according to any of embodiments 22-31, wherein said antigen is the spike protein from SARS-Cov-2 or a part thereof.
33. A vaccine kit comprising:
a first composition comprising an antigen, a TLR2 agonist and at least one pharmaceutically acceptable excipient, and
a second composition comprising vitamin A.
34. The vaccine kit according to embodiment 33, wherein said antigen is a protein or a multimer thereof, a peptide or a multimer thereof, an attenuated bacterium or an attenuated virus.
35. The vaccine kit according to any of embodiments 33-34, wherein said TLR2 agonist is the compound of

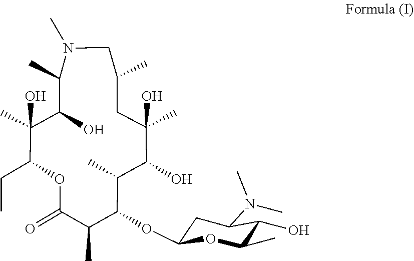

or a pharmaceutically acceptable salt thereof.
36. The vaccine kit according to any of embodiments 33-34, wherein said TLR2 agonist is an analogue of the compound of Formula (I), wherein said analogue is a compound of Formula (Ia) or a pharmaceutically acceptable salt, hydrate, solvate, tautomer, enantiomer or diastereomer thereof:

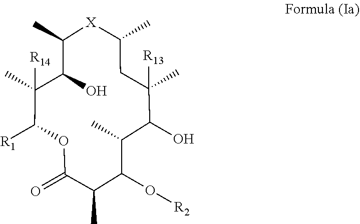

wherein X is selected from C═O, —NR₃CH₂—, —CH₂NR₃—, —NR₃(C═O)—, —(C═O)NR₃—, C═NOH, and —CH(OH)—, and R₂ is a sugar of Formula (II) or Formula (III):

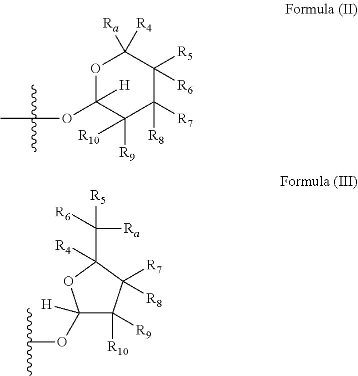

wherein R₁ is selected from an alkyl, heteroalkyl, cycloalkyl, aryl, and heteroaryl moiety,
wherein alkyl moiety is selected from C₁-C₈ alkyl groups that are optionally branched,
wherein heteroalkyl moiety is selected from C₁-C₆ alkyl groups that are optionally branched or substituted and that optionally comprise one or more heteroatoms,
wherein cycloalkyl moiety is selected from a C₁-C₆ cyclic alkyl groups that are optionally substituted and that optionally comprise one or more heteroatoms,
wherein aryl moiety is selected from optionally substituted C aromatic rings,
wherein heteroaryl moiety is selected from optionally substituted C₁-C₅ aromatic rings comprising one or more heteroatoms,
wherein heteroatoms are selected from O, N, P, and S,
wherein substituents, independently, are selected from alkyl, OH, F, Cl, NH₂, NH-alkyl, NH-acyl, S-alkyl, S-acyl, O-alkyl, and O-acyl,
wherein acyl is selected from C₁-C₄ optionally branched acyl groups,
wherein R₃ is selected from H and Me,
wherein R₄ is selected from H and Me,
wherein Rₐ is selected from H and CR₂₁R₂₂R₂₃,
wherein R₂₁, R₂₂, R₂₃, and R₅, R₆, R₇, R₃, R₉, and R₁₀, independently, are selected from H, Me, NR₁₁R₁₂, NO₂, and OR₁₁,
wherein R₂₃ together with R₄ in Formula (II), R₄ together with R₅ in Formula (II), R₅ together with R₇ in Formula (II), and R₇ together with R₉ in Formula (II), independently, may be joined to represent a bond to leave a double bond between the carbon atoms that each group is connected to,
wherein R₂₁ together with R₂₂, R₅ together with R₆, R₇ together with R₃, or R₉ together with R₁₀ may be replaced with a carbonyl,
wherein R₁₁ and R₁₂, independently, are selected from H and alkyl,
wherein R₁₃ is selected from H, OH, and OCH₃,
wherein R₁₄ is selected from H and OH,
and wherein one of R₅, R₆, R₇, R₃, R₉ or R₁₀ is selected from NR₁₁R₁₂ and NO₂,
with the proviso that when R₁ is Et, R₂ is a sugar of Formula (II), R₁₃ is H or OH, R₁₄ is H or OH, Ra is H, R₄ is Me, R₅ is H, R₆ is OH, R₇ is H, R₈ is NR₁₁R₁₂, R₉ is H, and R₁₀ is H, X may not be C═O.
with the proviso that when R₁ is Et, R₂ is a sugar of Formula (II), R₁₃ is H or OH, R₁₄ is H or OH, Ra is H, R₄ is Me, R₅ is OH, R₆ is H, R₇ is OH, R₃ is Me, R₉ is H, and R₁₀ is H, X may not be C═O.
with the proviso that when R₁ is Et, R₂ is a sugar of Formula (II), R₁₃ is H or OH, R₁₄ is H or OH, Ra is H, R₄ is Me, R₅ is OH, R₆ is H, R₇ is H, R₈ is NR₁₁R₁₂, R₉ is H, and R₁₀ is OH, X may not be C═O.
37. The vaccine kit according to embodiment 36, wherein said TLR2 agonist is selected from:

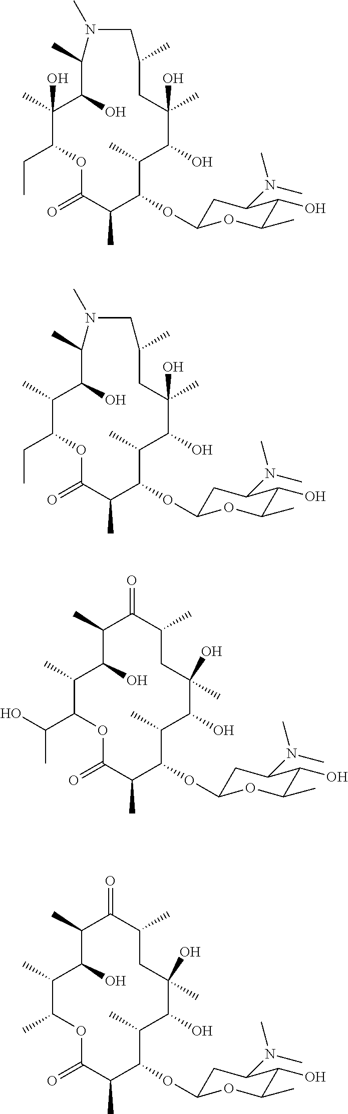 [SMILES: CC[C@H]1OC(=O)[C@H](C)[C@@H](OC2CC(N(C)C)C(O)C(C)O2)[C@H](C)[C@@H](O)[C@](C)(O)C[C@@H](C)CN(C)[C@H](C)[C@@H](O)[C@]1(C)O ; CC[C@H]1OC(=O)[C@H](C)[C@@H](OC2CC(N(C)C)C(O)C(C)O2)[C@H](C)[C@@H](O)[C@](C)(O)C[C@@H](C)CN(C)[C@H](C)[C@@H](O)[C@H]1C ; CC(O)C1OC(=O)[C@H](C)[C@@H](OC2CC(N(C)C)C(O)C(C)O2)[C@H](C)[C@@H](O)[C@](C)(O)C[C@@H](C)C(=O)[C@H](C)[C@@H](O)[C@H]1C ; CC1OC(O[C@H]2[C@H](C)[C@@H](O)[C@](C)(O)C[C@@H](C)C(=O)[C@H](C)[C@@H](O)[C@@H](C)[C@@H](C)OC(=O)[C@@H]2C)CC(N(C)C)C1O]

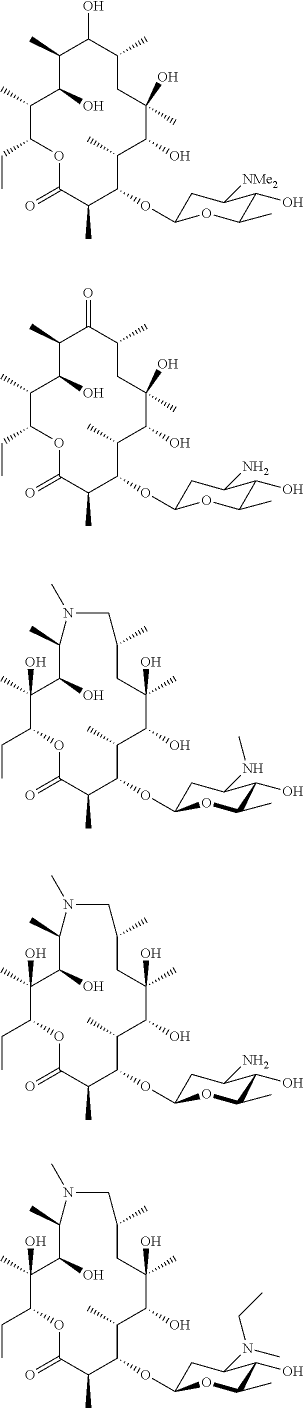

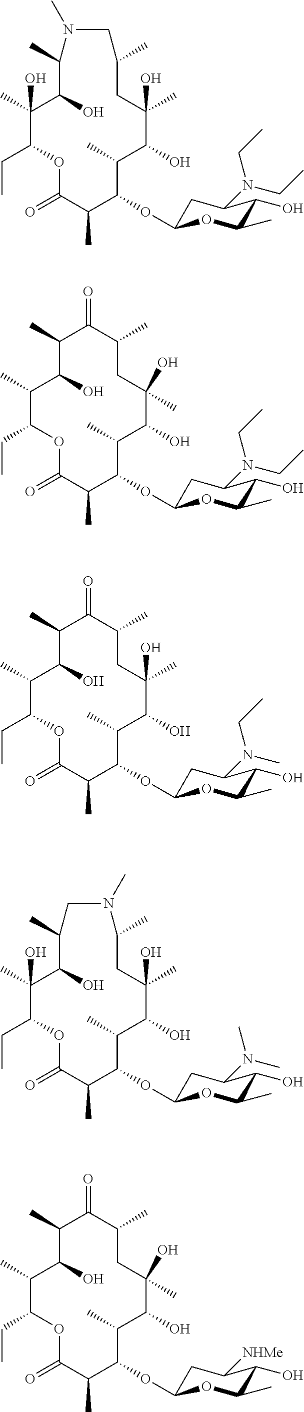

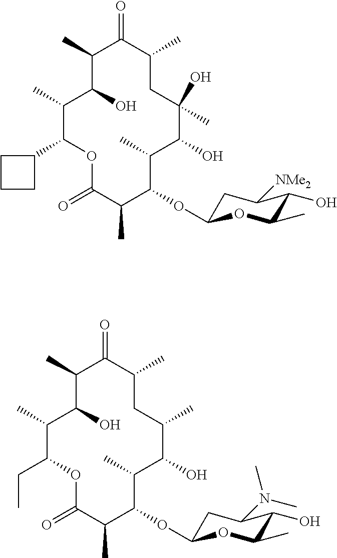

38. The vaccine kit according to any of embodiments 33-37, wherein said second composition comprises vitamin D.
39. The vaccine kit according to any of embodiments 33-37, wherein said vaccine kit comprises a third composition comprising vitamin D.
40. The vaccine kit according to embodiment 39, wherein said third composition is for oral administration.
41. The vaccine kit according to any of embodiments 33-40, wherein said first composition is for pulmonary or intranasal administration.
42. The vaccine kit according to any of embodiments 33-41, wherein said second composition is for oral administration.
43. The vaccine kit according to any of embodiments 33-42, wherein said antigen is attenuated SARS-Cov-2 or a component thereof.
44. The vaccine kit according to any of embodiments 33-43, wherein said antigen is the spike protein from SARS-Cov-2 or a part thereof.

### EXPERIMENTAL

TLR2 Assay

Samples and controls were tested in duplicate on recombinant HEK-293-TLR cell lines using a cell reporter assay at Invivogen using their standard assay conditions. These cell lines functionally over-express human TLR2 protein as well as a reporter gene which is a secreted alkaline phosphatase (SEAP). The production of this reporter gene is driven by an NFkB inducible promoter. The TLR reporter cell lines activation results are given as optical density values (OD).

20 μl of each test article were used to stimulate the hTLR2 reporter cell lines in a 200 μl of final reaction volume. Samples were tested in duplicate, with at least two concentrations tested—20 uM and 10 uM.

### Example 1—Generation of Compound 1

Generation of az-AG

Azithromycin aglycone was generated using methods described in the literature (Djokic, S., et al., 1988). In brief azithromycin is converted to azithromycin aglycone by the acidic removal of the 3-O and 5-O sugars. The 5-O amino sugar is first oxidised and pyrolyzed to facilitate cleavage.

Generation of Biotransformation Strains Capable of Glycosylating Erythromycin Agly-Cones (Erythronolides)

Generation of S. erythraea 18A 1 (pAES52)

pAES52, an expression plasmid containing angAI, angAII, angCVI, ang-orf14, angMIII, angB, angMI and angMII along with the actII-ORF4 pactI/III expression system (Rowe et al., 1998) was generated as follows.

The angolamycin sugar biosynthetic genes were amplified from a cosmid library of strain S. eurythermus ATCC23956 obtained from the American Type Culture Collection (Manassas, Virginia, USA). The biosynthetic gene cluster sequence was deposited as EU038272, EU220288 and EU232693 (Schell, 2008).

The biosynthetic gene cassette was assembled in the vector pSG144 as described pre-viously (Schell, 2008, ESI), adding sequential genes until the 8 required for sugar biosynthesis were obtained, creating plasmid pAES52.

pAES52 was transformed into strain 18A1 (WO2005054265).

Transformation of pAES52 into S. erythraea 18A1 pAES52 was transformed by protoplast into S. erythraea 18A1 using standard methods (Kieser et al 2000, Gaisser et al. 1997). The resulting strain was designated ISOM-4522, which is deposited at the NCIMB on 24 Jan. 2017 with Accession number: NCIMB 42718.

Generation of S. erythraea SGT2 (pAES54) pAES54, an expression plasmid containing angAI, angAII, angCV, ang-orf14, angMIII, angB, angMI and angMII along with the actII-ORF4 pactI/III expression system (Rowe et al., 1998) was generated as follows

The angolamycin sugar biosynthetic genes were amplified from a cosmid library of strain S. eurythermus ATCC23956 obtained from the American Type Culture Collection (Manassas, Virginia, USA). The biosynthetic gene cluster sequence was deposited as EU038272, EU220288 and EU232693 (Schell, 2008).

The biosynthetic gene cassette was assembled in the vector pSG144 as described pre-viously (Schell, 2008, ESI), adding sequential genes until the 8 required for sugar biosynthesis were obtained, creating plasmid pAES52.

Plasmid pAES54 was made by ligating the 11,541 bp SpeI-NheI fragment containing the actII-ORF4 pactI/III promotor system and the 8 ang genes was excised from pAES52 with the 5,087 bp XbaI-SpeI fragment from pGP9, containing an apramycin re-sistance gene, oriC, oriT for transfer in streptomycetes and phiBT1 integrase with attP site for integrative transformation. (The compatible NheI and XbaI sites were elimi-nated during the ligation.)

pAES54 was then transformed into S. erythraea SGT2 (Gaisser et al. 2000, WO2005054265).

Transformation of pAES54 into S. Erythraea SGT2

pAES54 was transferred by conjugation into S. erythraea SGT2 using standard methods. In brief, E. coli ET12567 pUZ8002 was transformed with pAES54 via standard procedures and spread onto 2TY with Apramycin (50 μg/mL), Kanamycin (50 μg/mL), and Chloramphenicol (33 μg/mL) selection. This plate was incubated at 37° C. overnight. Colonies from this were used to set up fresh liquid 2TY cultures which were incubated at 37° C. until late log phase was reached. Cells were harvested, washed, mixed with spores of S. erythraea SGT2, spread onto plates of R₆ and incubated at 28° C. After 24 hours, these plates were overlaid with 1 mL of sterile water containing 3 mg apramycin and 2.5 mg nalidixic acid and incubated at 28° C. for a further 5-7 days. Exconjugants on this plate were transferred to fresh plates of R₆ containing apramycin (100 μg/mL).

Alternative Biotransformation Strain

Alternatively, BIOT-2945 (Schell et al., 2008) may be used as the biotransformation strain, as this also adds angolosamine to erythronolides.

Biotransformation of Azithromycin Aglycone

Erlenmeyer flasks (250 mL) containing SV2 medium (40 mL) and 8 uL thiostrepton (25 mg/mL) were inoculated with 0.2 mL of spore stock of strain ISOM-4522 and incubated at 30° C. and shaken at 300 rpm with a 2.5 cm throw for 48 hours.

SV2 Media

| Ingredient | Amount |
|---|---|
| glycerol | 15 g |
| glucose | 15 g |
| soy peptone A3SC | 15 g |
| NaCl | 3 g |
| CaCO₃ | 1 g |
| RO water | To final volume of 1 L |

Pre-sterilisation pH adjusted to pH 7.0 with 10M HCl
Sterilised by autoclaving @ 121° C., −30 minutes

Sterile bunged falcon tubes (50 mL) containing EryPP medium (7 mL) were prepared and inoculated with culture from seed flask (0.5 mL per falcon tube) without antibiot-ics. The falcons were incubated at 30° C. and shaken at 300 rpm with a 2.5 cm throw for 24 hours.

ERYPP Medium

| Ingredient | Amount |
|---|---|
| toasted soy flour (Nutrisoy) | 30 g |
| glucose | 50 g |
| (NH₄)₂SO₄ | 3 g |
| NaCl | 5 g |
| CaCO₃ | 6 g |
| RO water | To final volume of 1 L |

Pre-sterilisation pH adjusted to pH 7.0 with 10M HCl
Sterilised in situ by autoclaving @ 121° C., 30 minutes
Post sterilisation 10 ml/L propan-1-ol added

After 24 hours, azithromycin aglycone (0.5 mM in DMSO, 50 uL) was added to each falcon tube and incubation continued at 300 rpm with a 2.5 cm throw for a further 6 days.

Isolation of Compound 1

Whole broth was adjusted to pH 9.5 and extracted twice with one volume of ethyl acetate. The organic layers were collected by aspiration following centrifugation (3,500 rpm, 25 minutes). The organic layers were combined and reduced in vacuo to reveal a brown gum that contained compound 1. This extract was partitioned between ethyl acetate (200 ml) and aqueous ammonium chloride (20 ml of a 50% concentrated solution). After separation, the organic layer was extracted with a further volume (200 ml) of the ammonium chloride aqueous solution. The combined aqueous layers were then adjusted to pH 9.0 with aqueous sodium hydroxide and then extracted twice with one volume equivalent of ethyl acetate. The organic layers were combined and reduced in vacuo to a brown solid. This extract was then applied to a silica column and eluted step wise (in 500 ml lots) with:

| Solvent | Hexanes | EtOAc | MeOH | Aq. NH₄OH |
|---|---|---|---|---|
| A | 0.499 | 0.499 | 0 | 0.002 |
| B | 0.250 | 0.748 | 0 | 0.002 |
| C | 0 | 0.998 | 0 | 0.002 |
| D | 0 | 0.988 | 0.01 | 0.002 |
| E | 0 | 0.978 | 0.02 | 0.002 |
| F | 0 | 0.968 | 0.03 | 0.002 |
| G | 0 | 0.958 | 0.04 | 0.002 |

compound 1 was predominantly in F and G. These solvents were combined and reduced in vacuo to yield a brown solid containing compound 1. This material was then purified by preparative HPLC (C18 Gemini NX column, Phenomenex with 20 mM ammonium acetate and acetonitrile as solvent). Fraction containing the target compound were pooled and taken to dryness followed by desalting on a C18 SPE cartridge.

### Example 2—Efficacy of COVID-19 Vaccine in Mice

The objective of the present study was to evaluate the efficacy of a novel COVID-19 vaccine in hACE2 transgenic mice.

Fifty-five female AC70 hACE2 transgenic mice were included in the study, granted by the regional animal ethics committee in Stockholm (2020-2021). Animals were divided into seven groups of 7 or 8 animals/group, to be immunized with vaccine, as follows:
Group 1: 10 μg Trimeric Spike+10 μg poly 1:C+40 μg Vitamin A; subcutaneous (n=7)
Group 2: No Vaccine (n=8)
Group 3: 100 μg Trimeric Spike+10 μg poly 1:C+40 μg Vitamin A, subcutaneous (n=8)
Group 4: 10 μg Trimeric Spike+10 μg poly 1:C+40 μg Vitamin A, intranasal (n=8)
Group 5: 80 μg Trimeric Spike+10 μg poly 1:C+40 μg Vitamin A, intranasal (n=8)
Group 6: 10 μg Trimeric Spike+10 μg poly 1:C+40 μg Vitamin A, intratracheal (n=8)
Group 7: 80 μg Trimeric Spike+10 μg poly 1:C+40 μg Vitamin A, intratracheal (n=8)

TABLE 1
Treatment groups

| | | Dose per | Dosing | | |
|---|---|---|---|---|---|
| Group | Compound | vaccination | Volume | Route | N |
| 1 | Test item | 10 μg Trimeric | 100 μL | Subcutaneous | 7 |
| | 2 + 5 | Spike | | | |
| | | 10 μg poly I:C | | | |
| | | 40 μg ATRA | | | |
| 2 | Vehicle | — | — | — | 8 |
| 3 | Test item | 100 μg Trimeric | 100 μL | Subcutaneous | 8 |
| | 1 + 5 | Spike | | | |
| | | 10 μg poly I:C | | | |
| | | 40 μg ATRA | | | |
| 4 | Test item | 10 μg Trimeric | 25 μL | Intranasal | 8 |
| | 4 + 5 | Spike | | | |
| | | 10 μg poly I:C | | | |
| | | 40 μg ATRA | | | |
| 5 | Test item | 80 μg Trimeric | 25 μL | Intranasal | 8 |
| | 3 + 5 | Spike | | | |
| | | 10 μg poly I:C | | | |
| | | 40 μg ATRA | | | |
| 6 | Test item | 10 μg Trimeric | 25 μL | Intratracheal | 8 |
| | 4 + 5 | Spike | | | |
| | | 10 μg poly I:C | | | |
| | | 40 μg ATRA | | | |
| 7 | Test item | 80 μg Trimeric | 25 μL | Intratracheal | 8 |
| | 3 + 5 | Spike | | | |
| | | 10 μg poly I:C | | | |
| | | 40 μg ATRA | | | |

Poly I:C was obtained from Invitrogen (vac-pic).
SARS-COV-2 trimeric spike protein was obtained from Icosagen.

Animals were weighed and immunised on Day 0 and 14 (Groups 3-7) or 15 (Group 1). On Day 28, animals were inoculated with 1.875×10⁵ TCID50 SARS-CoV-2 via intranasal administration. Animals were weighed and monitored for changes in health status daily until Days 38-39, whereafter they were euthanised. Animals that had lost 20% of their body weight, or showed a severe decline in health status, were euthanised pre-term, according to the ethical permit governing this experiment.

Blood samples for isolation of serum were acquired on Day−3, Day 14, Day 28 and at termination. Following blood sampling at termination, animals were euthanised, and bronchioalveolar lavage was performed and fluid (BAL) was collected. Spleen, lung and trachea were excised and a section of spleen, lung (lower airway) and trachea (up-per airway) were saved in RNALater and TRIzol for analysis of viral titres. Lung and skull (for brain and nasopharyngeal tissues) were saved in 4% formaldehyde for histopathological analysis.

One animal (ID 343, Group 3) received an imperfect subcutaneous dose on Day 0. One animal (ID 366, Group 6) did not receive the first immunisation, due to lack of test item. Three animals (IDs 371, 373 and 375, Group 7) died following the first immunisation: one animal died due to an overdose of anaesthetic and the remaining two animals were euthanised due to complications caused by the intratracheal administration tech-nique. One animal (ID 341, Group 3) was found dead following the second immunisation, due to lack of oxygen caused by failure to properly insert the IsoCage into the rack.

Vaccine administration per se did not overly affect animal body weights. A small decline in body weight was evident for animals in Group 7 between Days 0 and 14; however, all groups showed a general increase in body weight following the second vaccination.

Administration of SARS-CoV-2 in non-vaccinated animals resulted in a significant decline in body weight; animals had dropped to 85.7±0.7% of their pre-inoculation body weights by Day 32. Vaccination significantly affected infection-induced decline in body weight, with no marked decline in relative body weights in all vaccination groups. However, one animal in Group 6 (ID 366) demonstrated a marked decline in body weight, similar to that of non-vaccinated animals. As described above, this animal had only received one immunisation (Day 14).

Differences in body weight were additionally evident between vaccinated groups. Relative body weights for Groups 1 (low dose subcutaneous) and 4 (low dose intranasal) were significantly lower than those of Group 5, 6 and 7.

Vaccine administration did not overtly affect animal health status and had no observa-ble effect on respiratory function. In comparison, inoculation with SARS-CoV-2 was associated with a deterioration in health status, from four days after inoculation. Animals presented with hunched posture, piloerection and decreased movements. Two animals showed signs of aggression and two animals had abnormal motor behaviour, namely standing on their hind legs and rocking back and forth. Due to the deterioration in health status, as well as body weight loss, animals in Group 2 were euthanised on Day 32.

Vaccinated animals showed few changes in overt health status. One animal in Group 6 (ID 366) presented with symptoms on Day 32 and was consequently euthanised. Three animals in Group 1 were euthanised on Days 32 or 34, due to presentation of hunched posture, piloerection, increased movement, rigidity and tremor. No overt symptoms were evident for the remaining vaccinated animals.

Vaccine administration significantly improved survival. Median survival for non-vaccinated animals was 4 days, which was significantly different to survival of animals in all other groups. Animals in Group 1 had a median survival of 6 days; remaining vaccinated groups had undefined survival, as animals were euthanised on termination day and not due to health status decline.

Circulating IgG titres against Spike (Wuhan) and Spike receptor-binding domain, RBD, (South Africa and U.K.) were detected in all vaccinated groups on Day 28; increases were dose-dependent, with animals receiving high dose Spike showing stronger immu-nological responses. In comparison, IgA titres against Spike and RBD were only detected in groups that were vaccinated via the intranasal or intratracheal route. In particular, intranasal administration was associated with a significant increase in IgA titres. In comparison to IgG, no dose-dependency was evident.

IgG and IgA antibody titres were detected in terminal BAL samples of vaccinated animals. Strongest responses were evident for animals vaccinated via the intratracheal and intranasal route, and a dose-dependent response was evident. IgG responses were predominant, particularly against Spike and RBD (U.K.).

Neutralising antibody titres were observed at varying levels in the broncheoalveolar lavage of vaccinated animals. Subcutaneous administration was associated with the low-est level of neutralising antibodies, with only 3 animals showing low or partial titres. Animals vaccinated via the intranasal or intratracheal route showed higher levels of neutralising antibody titres; highest levels were detected following high dose intranasal administration.

SARS-CoV-2 virus was detectable in BAL in all non-vaccinated control animals, indica-tive of a successful infection. Low viral titres were detected in three animals that had received subcutaneous vaccination but were undetectable in animals that were vaccinated via intranasal or intratracheal administration.

Histopathological analysis revealed inflammatory changes in the respiratory tract in all groups. Of interest, however, inflammatory cell infiltration in the lower respiratory tract (trachea, carina and lungs) was either not detected or detected at a lower severity in non-vaccinated animals. Perivascular to parabronchial and alveolar to interstitial inflammatory cell infiltration was observed to a higher degree in Group 1 (LD s.c.), and slightly higher in Groups 5 (HD i.n.) and Group 7 (HD i.t.). Only minimal changes were observed in non-vaccinated controls. In comparison, inflammatory cell infiltration and decreased lumen in arterioles, as well as bronchiolar debris was observed to the highest degree in groups which had the test item injected subcutaneously. These changes were observed to a lesser degree in animals that received intranasal or intratracheal immunisation and were not observed in non-vaccinated controls. As inflammation was minimal in non-vaccinated controls, inflammatory changes may be evidence of a vac-cine-driven anti-viral immune response.

Notably, inflammatory changes were also observed in the central nervous system, namely the striatum, which may explain the abnormal motor behaviours observed in some animals. Neuronal necrosis in the piriform cortex, as well as perivascular inflammatory cell infiltration was observed in the meninges and parenchyma in non-vaccinated animals, and animals vaccinated via the subcutaneous route. These changes were not observed in remaining groups, suggesting that intratracheal or intranasal administration of vaccine prevents viral infiltration into the CNS.

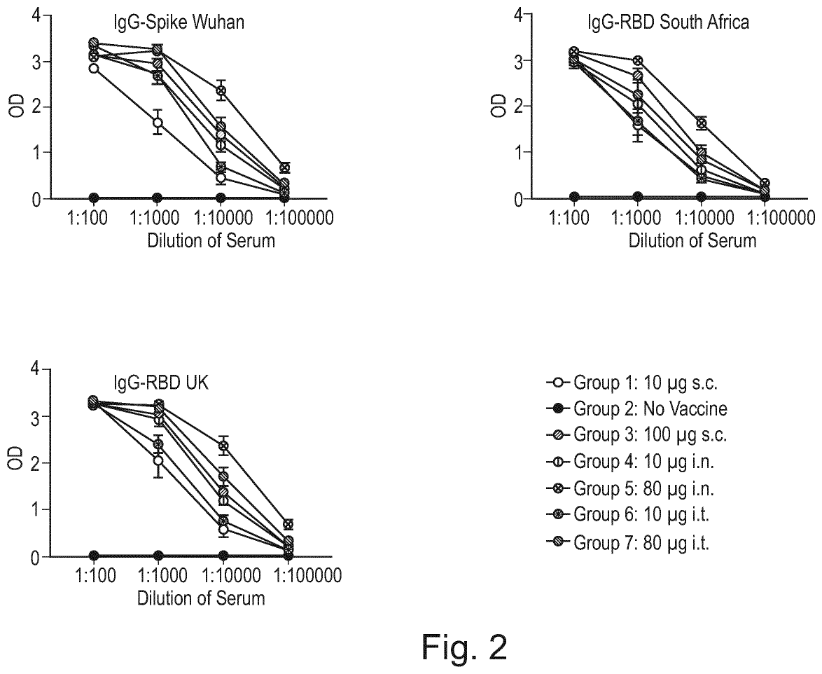

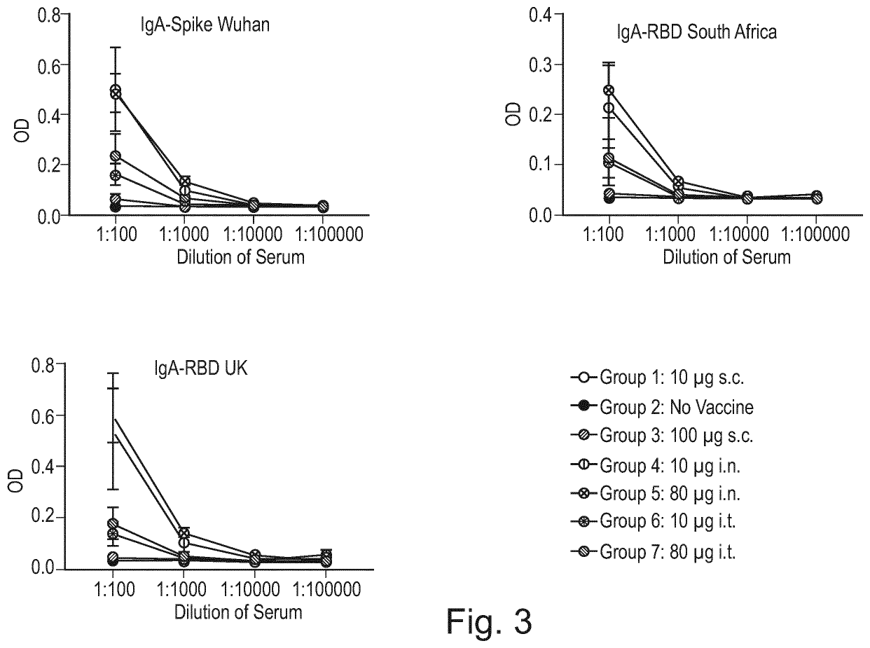

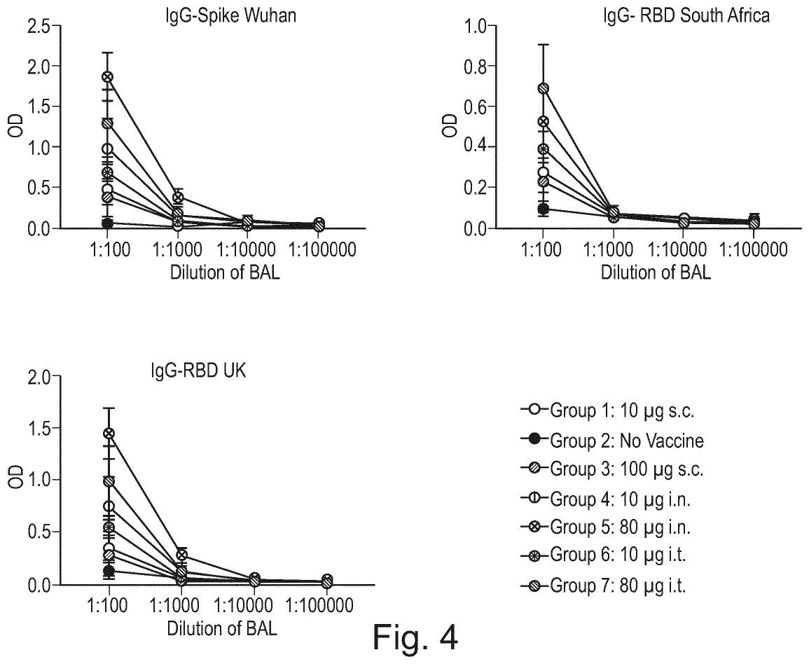

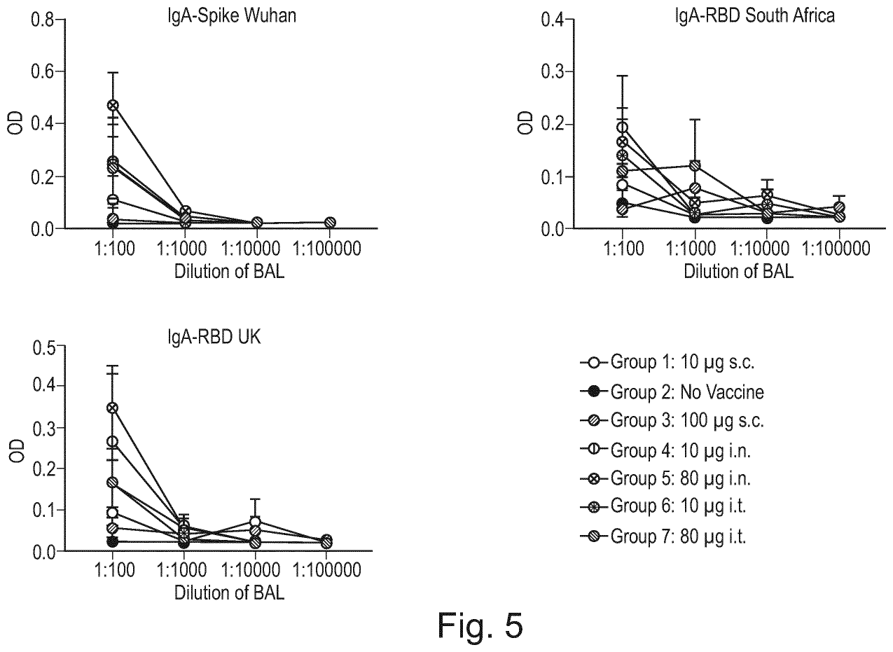

Results from the study are shown in the below Tables 2-3 and in FIGS. 2-5.

TABLE 2
Descriptive statistics of absolute body weights (g), showing mean, standard error of the mean (SEM)
and number of animals (N). Animals were vaccinated on Days 0 and 14 and infected on Day 28.

| | 10 μg s.c. | | | No Vaccine | | | 100 μg s.c. | | | 10 μg i.n. | | | 80 μg i.n. | | | 10 μg i.t. | | | 80 μg i.t. | | |
|---|---|---|---|---|---|---|---|---|---|---|---|---|---|---|---|---|---|---|---|---|---|
| Day | Mean | SEM | N | Mean | SEM | N | Mean | SEM | N | Mean | SEM | N | Mean | SEM | N | Mean | SEM | N | Mean | SEM | N |
| 0 | 19.8 | 0.5 | 7 | 21.3 | 0.6 | 8 | 22.3 | 0.4 | 8 | 21.7 | 0.7 | 8 | 20.4 | 0.4 | 8 | 22.1 | 0.3 | 8 | 23.0 | 0.4 | 8 |
| 14 | 20.3 | 0.4 | 7 | 21.2 | 0.6 | 8 | 21.6 | 0.8 | 8 | 21.4 | 0.8 | 8 | 20.9 | 0.3 | 8 | 21.4 | 0.4 | 8 | 21.5 | 0.5 | 5 |
| 28 | 21.6 | 0.6 | 7 | 22.9 | 0.8 | 8 | 23.1 | 0.5 | 7 | 23.5 | 0.6 | 8 | 21.2 | 0.4 | 8 | 22.6 | 0.3 | 8 | 22.6 | 0.5 | 5 |
| 29 | 21.2 | 0.5 | 7 | 22.5 | 0.8 | 8 | 22.8 | 0.5 | 7 | 22.7 | 0.6 | 8 | 20.7 | 0.4 | 8 | 22.1 | 0.4 | 8 | 22.1 | 0.5 | 5 |
| 30 | 21.7 | 0.5 | 7 | 23.1 | 0.7 | 8 | 23.0 | 0.5 | 7 | 23.5 | 0.6 | 8 | 21.5 | 0.4 | 8 | 22.7 | 0.4 | 8 | 22.7 | 0.7 | 5 |
| 31 | 21.6 | 0.5 | 7 | 22.6 | 0.7 | 8 | 23.2 | 0.5 | 7 | 23.4 | 0.7 | 8 | 21.3 | 0.4 | 8 | 22.8 | 0.4 | 8 | 22.8 | 0.6 | 5 |
| 32 | 21.0 | 0.6 | 7 | 19.6 | 0.7 | 8 | 23.1 | 0.5 | 7 | 23.3 | 0.7 | 8 | 21.3 | 0.4 | 8 | 22.4 | 0.6 | 8 | 23.0 | 0.7 | 5 |
| 33 | 19.8 | 0.9 | 7 | — | — | 0 | 22.9 | 0.5 | 7 | 23.3 | 0.7 | 8 | 21.4 | 0.4 | 8 | 23.2 | 0.5 | 7 | 23.1 | 0.7 | 5 |
| 34 | 19.6 | 1.6 | 4 | — | — | 0 | 22.4 | 0.6 | 7 | 23.0 | 0.7 | 8 | 21.5 | 0.4 | 8 | 23.0 | 0.5 | 7 | 23.1 | 0.6 | 5 |
| 35 | 22.9 | 0.2 | 2 | — | — | 0 | 23.3 | 0.5 | 6 | 23.5 | 0.8 | 8 | 21.7 | 0.4 | 8 | 23.2 | 0.5 | 7 | 23.1 | 0.6 | 5 |
| 36 | 22.9 | 0.2 | 2 | — | — | 0 | 23.1 | 0.6 | 6 | 23.8 | 0.8 | 8 | 21.5 | 0.3 | 8 | 23.4 | 0.5 | 7 | 22.9 | 0.8 | 5 |
| 37 | 22.9 | 0.1 | 2 | — | — | 0 | 23.1 | 0.7 | 6 | 23.6 | 0.7 | 8 | 21.6 | 0.4 | 8 | 23.3 | 0.4 | 7 | 23.1 | 0.7 | 5 |
| 38 | 22.6 | 0.2 | 2 | — | — | 0 | 23.1 | 0.5 | 6 | 23.5 | 0.7 | 8 | 21.8 | 0.5 | 8 | 23.3 | 0.4 | 7 | 23.3 | 0.7 | 5 |

TABLE 3
Descriptive statistics of relative body weights (%), showing mean, standard error
of the mean (SEM) and number of animals (N), following infection with SARS-CoV-2.

| | 10 μg s.c. | | | No Vaccine | | | 100 μg s.c. | | | 10 μg i.n. | | | 80 μg i.n. | | | 10 μg i.t. | | | 80 μg i.t. | | |
|---|---|---|---|---|---|---|---|---|---|---|---|---|---|---|---|---|---|---|---|---|---|
| Day | Mean | SEM | N | Mean | SEM | N | Mean | SEM | N | Mean | SEM | N | Mean | SEM | N | Mean | SEM | N | Mean | SEM | N |
| 28 | 100.0 | 0.0 | 7 | 100.0 | 0.0 | 8 | 100.0 | 0.0 | 7 | 100.0 | 0.0 | 8 | 100.0 | 0.0 | 8 | 100.0 | 0.0 | 8 | 100.0 | 0.0 | 5 |
| 29 | 98.1 | 0.5 | 7 | 98.1 | 0.4 | 8 | 98.8 | 0.5 | 7 | 96.6 | 0.5 | 8 | 97.6 | 0.4 | 8 | 97.8 | 0.4 | 8 | 98.1 | 0.4 | 5 |
| 30 | 100.3 | 0.9 | 7 | 100.8 | 0.6 | 8 | 99.7 | 0.8 | 7 | 99.8 | 1.0 | 8 | 101.3 | 0.7 | 8 | 100.3 | 0.6 | 8 | 100.3 | 1.0 | 5 |
| 31 | 99.9 | 0.7 | 7 | 98.7 | 1.2 | 8 | 100.6 | 0.5 | 7 | 99.7 | 1.0 | 8 | 100.5 | 1.0 | 8 | 100.9 | 0.7 | 8 | 101.0 | 1.0 | 5 |
| 32 | 97.0 | 1.9 | 7 | 85.7 | 0.7 | 8 | 100.0 | 0.7 | 7 | 99.3 | 0.8 | 8 | 100.4 | 0.8 | 8 | 99.2 | 2.4 | 8 | 101.7 | 0.8 | 5 |
| 33 | 91.6 | 3.0 | 7 | — | — | 0 | 99.3 | 1.2 | 7 | 99.2 | 1.2 | 8 | 101.0 | 0.9 | 8 | 89.6 | 12.8 | 8 | 102.2 | 0.9 | 5 |
| 34 | 69.6 | 23.7 | 4 | — | — | 0 | 97.2 | 2.1 | 7 | 98.0 | 1.1 | 8 | 101.4 | 0.8 | 8 | 89.1 | 12.8 | 8 | 102.2 | 0.9 | 5 |
| 35 | 102.0 | 0.2 | 2 | — | — | 0 | 101.2 | 0.4 | 6 | 99.7 | 1.4 | 8 | 102.1 | 1.2 | 8 | 89.7 | 12.9 | 8 | 102.1 | 1.6 | 5 |
| 36 | 101.8 | 1.8 | 2 | — | — | 0 | 100.5 | 0.8 | 6 | 101.1 | 1.1 | 8 | 101.6 | 1.0 | 8 | 90.4 | 13.0 | 8 | 101.4 | 1.7 | 5 |
| 37 | 102.0 | 1.6 | 2 | — | — | 0 | 100.4 | 1.2 | 6 | 100.2 | 0.8 | 8 | 102.0 | 1.3 | 8 | 90.1 | 12.9 | 8 | 102.5 | 1.5 | 5 |
| 38 | 100.7 | 0.2 | 2 | — | — | 0 | 100.5 | 0.7 | 6 | 99.7 | 1.1 | 8 | 102.9 | 1.2 | 8 | 90.2 | 13.0 | 8 | 103.0 | 1.1 | 5 |

In summary, Intranasal inoculation with 1.875×10⁵ TCID50 SARS-CoV-2 resulted in a decrease in body weight and a deterioration in health status, resulting in pre-term eu-thanasia within four days of infection. This was associated with increased viral titres in the lower respiratory tract. Intranasal and intratracheal administration of trimeric spike (10-80 μg), poly 1:C (10 μg) and all trans retinoic acid (ATRA) (40 μg) had no overall effect on health status. Vaccinated animals showed a dose-dependent serological response, with systemic and local production of IgG and IgA antibodies against Spike and RBD, as well as local production of neutralising antibodies. This was associated with lack of viral replication in the lungs, inhibition of SARS-CoV-2-driven encephalitis, and prevention of covid-19 disease progression.

In conclusion, the study showed that intranasal and intratracheal vaccination with SARS-CoV-2 Trimeric Spike protein (10-80 μg), poly 1:C (10 μg) and vitamin A, on two occasions, fully protects against SARS-CoV-2 infection at 1.875×10⁵ TCID50.

### Example 3—COVID-19 Vaccine Using Spike Protein Coupled to Beads Together with AdJuvant and Vitamin A

The objective of this study was to assess the immunogenicity of a novel vaccine against COVID-19 in BALB/c mice.
Test Item 1: SARS-CoV-2 spike protein and the compound of Formula (I).
Test Item 2: SARS-CoV-2 spike protein.
Test Item 3: Calcitriol (Vitamin D), ATRA (Vitamin A) mix. The mix was prepared and handle carefully as it is very light sensitive. Concentration of Test Item 3 is 100 μg/mL Calcitriol and 20 mg/mL ATRA.

Twenty female BALB/c mice of 6-7 weeks age were weighed and divided into four groups of five animals per group as follows:

TABLE 4

| Group | Compound | Dosing (μL) | Route | N |
|---|---|---|---|---|
| 1 | Test Item 1 + Test Item 3 | 25 | Subcutaneous | 5 |
| | Test Item 3 | 100 | Intraperitoneal | |
| 2 | Test Item 1 + Test Item 3 | 25 | Intranasal | 5 |
| | Test Item 3 | 100 | Intraperitoneal | |
| 3 | Test Item 1 | 25 | Intranasal | 5 |
| 4 | Test Item 2 + Test Item 3 | 25 | Intranasal | 5 |
| | Test Item 3 | 100 | Intraperitoneal | |

Groups 1, 2 and 4 (i.p. injection, 100 μL×15 animals) to administer 100 ng Calcitriol and 20 μg ATRA per mouse.

Groups 1 and 2 (s.c. or i.n. administration, 25 μL×10 animals) to administer 100 ng Calcitriol and 20 μg ATRA per mouse.

Group 4 (i.n. administration, 25 μL×5 animals) to administer 100 ng Calcitriol and 20 pg ATRA per mouse.

Animals were immunised by subcutaneous or intranasal delivery on days 0, 10 and 20. Group 1, 2 and 4 were just before immunisation intraperitoneally injected 20 microgram ATRA and 100 nanogram Calcitriol (Test Item 3). Blood samples (150 μL) for isolation of serum and subsequent serological assessment were taken on day 0 (before immunisation), 10, 20 and 30. The blood samples were inverted 10 times, left at room temper-ature for 30 minutes and then centrifuged at 2000×g for 10 minutes at 4° C. Serum were then aliquoted into Eppendorf-tubes and stored at −20° C. until further analysis.

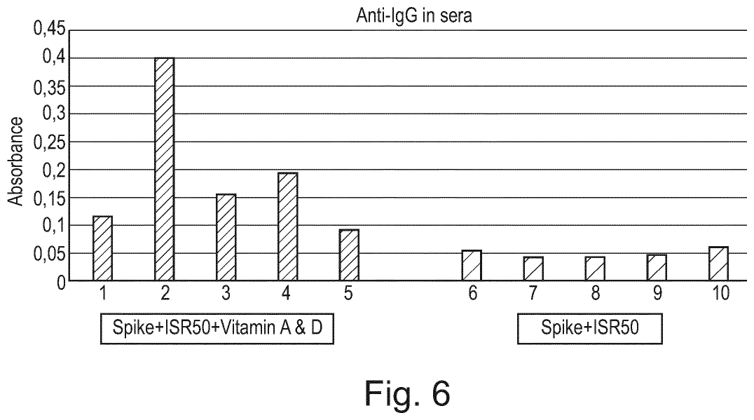

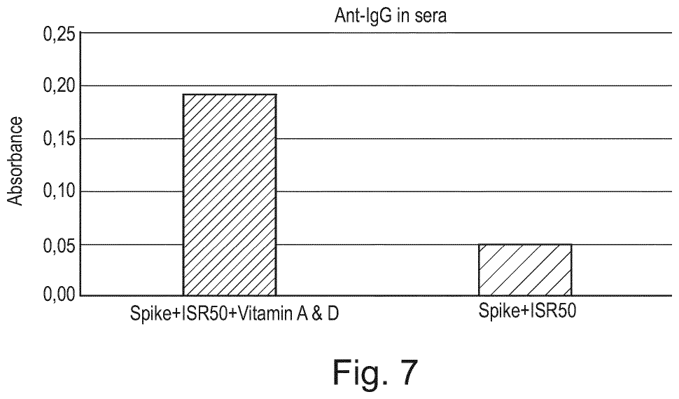

The anti-IgG response from sera collected at Day 30 from groups 2 (Spike, ISR50 and Vitamin A & D) and 3 (Spike and ISR50) showed a positive effect from administration of also Vitamin A and D. FIGS. 6 and 7 show the anti-IgG in sera from the animals of the groups (individual mice and average, respectively).

### REFERENCES

Kieser et al 2000 Practical Streptomyces Genetics, Published by the John Innes Foundation
Gaisser et al., 1997 Analysis of seven genes from the eryAl-eryK region of the erythromycin biosynthetic gene cluster in Saccharopolyspora erythraea. Mol Gen Genet., 1997 Oct.; 256(3):239-51.
Gaisser et al., 2000 A defined system for hybrid macrolide biosynthesis in Saccharopolyspora erythraea Mol. Micro., 2000; 36(2):391-401
Schell et al., 2008 Engineered biosynthesis of hybrid macrolide polyketides containing D-angolosamine and D-mycaminose moieties Org. Biomol. Chem., 2008; 6:3315-3327 Djokic, S., et al., Erythromycin Series. Part 13. Synthesis and Structure Elucidation of 10-Dihydro-10-deoxo-11-methyl-11-azaerythromycin A J. Chem. Res. (S), 1988; 5:152-153
Rowe et al., 1998 Construction of new vectors for high-level expression in actinomy-cetes. Gene. 1998 Aug. 17; 216(1):215-23.

All references referred to in this application, including patent and patent applications, are incorporated herein by reference to the fullest extent possible.

1. A method for vaccinating a patient in need thereof against respiratory viruses, comprising:
pulmonally or intranasally administering to the patient a first composition comprising an antigen, a TLR2 agonist, and at least one pharmaceutically acceptable excipient; and
orally administering to the patient vitamin A, wherein the vitamin A is administered at least once within three days before or after the administration of the first composition.

2. The method according to claim 1, wherein the antigen in the first composition is a protein or a multimer thereof, a peptide or a multimer thereof, an attenuated bacterium, or an attenuated virus.

3. The method according to claim 1, wherein the TLR2 agonist in the first composition is the compound of Formula (I): 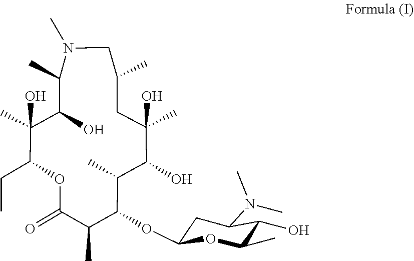
or is a pharmaceutically acceptable salt thereof.

4. The method according to claim 1, further comprising orally administering vitamin to the patient either before, at the same time, or within 3 days from the administration of the first composition.

5. The method according to claim 4, wherein the vitamin D is administered in the period between one week before the administration of the first composition and two days after the administration of the first composition.

6. The method according to claim 1, wherein the antigen in the first composition is attenuated SARS-Cov-2 or a component thereof.

7. The method according to claim 1, wherein the antigen in the first composition is the spike protein from SARS-Cov-2 or a part thereof.

8. The method according to claim 1, wherein the vitamin A is administered at least once in the period between one day before the administration of the first composition and two days after the administration of the first composition.

9. A vaccine kit for vaccinating against respiratory viruses, comprising:
a first composition comprising an antigen, a TLR2 agonist, and at least one pharmaceutically acceptable excipient; and
a second composition comprising vitamin A.

10. The vaccine kit according to claim 9, wherein the first composition is for pulmonary or intranasal administration.

11. The vaccine kit according to claim 9, wherein the antigen in the first composition is the spike protein from SARS-Cov-2 or a part thereof.

12. The vaccine kit according to claim 9, wherein the TLR2 agonist in the first composition is the compound of Formula (I): 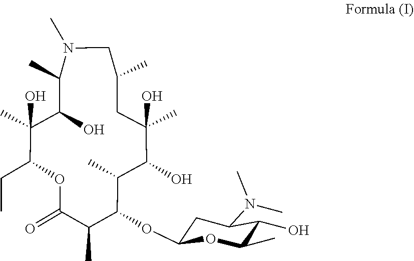
or is a pharmaceutically acceptable salt thereof.

13. A dosage regimen for vaccinating a patient in need thereof against respiratory viruses, comprising:
a first composition comprising an antigen, a TLR2 agonist, and at least one pharmaceutically acceptable excipient, wherein the first composition is formulated for pulmonary or intranasal administration to the patient; and
vitamin A, wherein the vitamin A is formulated for oral administration to the patent.

14. The dosage regimen according to claim 13, wherein the antigen is a protein or a multimer thereof, a peptide or a multimer thereof, or an attenuated virus.

15. The dosage regimen according to claim 13, wherein the TLR2 agonist is the compound of Formula (I): 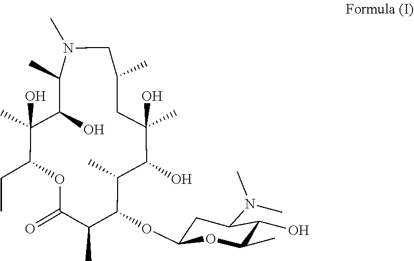
or is a pharmaceutically acceptable salt thereof.

16. The dosage regimen according to any claim 13, further comprising vitamin D, wherein the vitamin D is formulated for oral administration to the patient.

17. (canceled)

18. The dosage regimen according to claim 13, wherein the antigen is attenuated SARS-Cov-2 or a component thereof.

19. The dosage regimen according to claim 13, wherein the antigen is the spike protein from SARS-Cov-2 or a part thereof.

20-28. (canceled)
